SEQUENCE LISTING (22 sequences)

SEQ ID NO: 1 - protein, 634 aa, Aphis gossypii
MMIKLYCLYAILWLSVTIVWRPVLSAGTKKYSEIEASKYLDNANYALTEWTNRVIHANWNWLTNLTNENAEKKIAINLEFSKFLKSMWVETVKYPWSTYKDPDIKRQFKLMSVLGTDALPEDKLKKLDEIVSAMESLYGRATIPEYGDNNLNRTLSLEPDINDILDKSTDVNELKHVWVQWREATGKKIRPMYAEYVKLSNEAARLNNYTDNAEFWIRGYDVDDFRPRMEHLWNQIKPLYLQIHAYVRRKLWELYGSSVITRRGPIPAHLLGDMWAQSWERLDDFTRPYPTIDDVNPTSAMINQNYTPKKMFKVAEEFFTSLNLSAMPQTFWEKSILEKPNGRDLVCHASAWDFYDSNDFRIKQCTSVNFMDFITAHHEMGHIQYFLQYKDLPFIYRDGANEGFHEAIGDTIALSVSTPKHLHKIGLLPKTSRTYEADINYLYKIGLDKVVFLPFGYLMDLWRWNVFKGLTTEDQYNCDWWKLKYSYQGIEPPVTRTENDFDPGSKYHIVGNVPYIRYFVSYIVQFQFHQALCEKADQFDPKNPTSKPLHECDIYQSKNAGNAFKDMLKLGSSKPWFDAMELLTGQREMDARPLLNYFNPLYEWLKNENKRTGEHLGWETNKKICFKKDETSQP

SEQ ID NO: 2 - DNA, 1905 bp, Aphis gossypii
  CDS (1)..(1902)
atgatgataaaattatattgtttgtatgctatattatggctatctgttacgattgtttggcgaccagtactatctgctggaacaaaaaagtattctgaaattgaagcttccaaatatttggacaacgccaattacgcattgactgaatggacaaaccgagtcatacatgccaattggaattggttaacaaacttaactaatgaaaatgctgaaaaaaagatcgccattaatttggaatttagcaagttcttaaagagtatgtgggtagaaactgtgaaatatccatggtcaacatacaaagacccggatatcaaaagacagtttaaattaatgtcagtattaggaacagatgctttacccgaagacaaattgaaaaaactagatgaaatcgtttcggccatggaaagtttgtacggcagagcaaccattcctgagtatggcgataacaatttaaatcgaactttgagtttagaaccagatataaacgacatattggataaaagtacggatgtaaatgaactgaaacatgtatgggtacaatggagagaagccacaggaaaaaaaattcgacctatgtatgcggaatatgtaaaattatctaacgaagcggcgagattaaacaattacacagataatgcagaattttggattcgaggatacgatgtcgacgactttcggccgcgaatggaacatttatggaatcaaatcaaacctctatacttgcagatacacgcctacgtgcgtagaaagctatgggagctatacggtagctcagtgatcacaagaagaggacctataccagcacatctgctcggtgatatgtgggcgcagtcgtgggaacgtttggatgactttactcgaccttacccaactatcgatgatgtaaatcccacttcagccatgataaatcagaattacactccgaaaaagatgtttaaagtagctgaagaatttttcacatcgttaaacttgagcgcaatgccccaaactttctgggaaaaatctattttggagaagccaaatggtcgcgatctggtatgccatgcatccgcttgggacttttatgattctaatgactttagaattaaacaatgtacatcagtaaattttatggattttataactgcccatcatgagatgggacacatccaatatttcttacaatataaagacctgccgttcatataccgcgatggagctaacgaaggtttccacgaagcgatcggagatacaatcgctttgtctgtatcaacaccaaagcacttacataagattggtttgttacctaaaacgagtcgcacatatgaagccgatataaattatttgtacaagatcggattggacaaagtagtttttttaccttttgggtatttaatggatctatggaggtggaacgtattcaagggactcacgacggaagatcaatacaattgtgattggtggaaactcaaatattcttatcagggaatcgagccaccggtgaccagaactgagaacgattttgacccgggatccaaataccatatcgttgggaacgtgccatatattagatactttgtgagttacatcgtacaatttcaattccatcaagcattatgcgagaaagctgaccagtttgatcctaaaaatccaacaagtaaaccattacatgaatgcgacatttatcaaagcaaaaatgccggaaacgctttcaaagacatgttaaaattaggatcttcgaaaccgtggtttgatgctatggaactactaactggtcagagggaaatggacgctagacctttgttgaactatttcaaccctctatacgaatggcttaaaaatgaaaataaaagaactggagaacatcttggctgggaaacaaataaaaaaatttgctttaagaaggatgaaacaagtcaaccttaa

SEQ ID NO: 3 - DNA, 35 bp, Artificial Sequence
  Description of Artificial Sequence Synthetic primer
gaattcgcaccatgatgataaaattatattgtttg

SEQ ID NO: 4 - DNA, 33 bp, Artificial Sequence
  Description of Artificial Sequence Synthetic primer
ctcgagttaaggttgacttgtttcatccttctt

SEQ ID NO: 5 - DNA, 35 bp, Artificial Sequence
  Description of Artificial Sequence Synthetic primer
caaaaatgccggaaacgctttcaaagacatgttaa

SEQ ID NO: 6 - DNA, 56 bp, Artificial Sequence
  Description of Artificial Sequence Synthetic primer
ctcgagttagtgatggtgatggtgatgaggttgacttgtttcatccttcttaaagc

SEQ ID NO: 7 - DNA, 19 bp, Artificial Sequence
  Description of Artificial Sequence Synthetic oligonucleotide linker for ligation
gtccgaaacctcgagccgg

SEQ ID NO: 8 - DNA, 20 bp, Artificial Sequence
  Description of Artificial Sequence Synthetic oligonucleotide linker for ligation
aattccggctcgaggtttcg

SEQ ID NO: 9 - DNA, 17 bp, Artificial Sequence
  Description of Artificial Sequence Synthetic primer
caggaaacagctatgac

SEQ ID NO: 10 - DNA, 17 bp, Artificial Sequence
  Description of Artificial Sequence Synthetic primer
gttttcccagtcacgac

SEQ ID NO: 11 - DNA, 41 bp, Artificial Sequence
  Description of Artificial Sequence Synthetic primer
gtttcagttcatttacatccgtacttttatccaatatgtcg

SEQ ID NO: 12 - DNA, 1108 bp, Blattella germanica
  CDS (3)..(1106)
ttaccaagagcctggaccatgaagaattgaaacatgtgtgggtggaatggaggaaggtctctggagagaaatgcagaggactttttgagcattatatcgcactcagcaatgaggctgccacacttaacaatttcaccaacaacgctgaattttggttaagagattatgaagcacctgatttcaaagagcaagtatctaaactttgggaaaaaatagaaccactatacttgcagattcatgcatatgtcaggaggaagctaagagagacgtatggagaggagctcgtgaaaccacgtggaccaataccagcwcatcttttaggaaatatgtggtcccagacatggaatagtattctggatatttcaacacccttccctggaaaagaagcaatagatgtcacccctcaaatggtcaaacagggatacactcctttgaagatgttcaaactatcagaacaattcttcacatctctcaaccttagtgcaatgcccgaaatgttctgggagaagtcaattttgcagaaaccttcagatagagaacttgtttgtcatgcttctgcatgggacttctatgacggaaaagatttcaggatcaaacaatgtacgagggttaacatggatgatcttttcactgcacatcatgagatgggccacatccagtatttccttcaatataaagatcagcccacagtctacaaggaaggtgcyaatccwggwttycaygargcwgtmggwgacgtcatggctttatcagtttcaactccaaagcatttgaggaagattggtcttttggaagagagtgataaaggagaagactatgaggccactatcaattacttgttcctccaagggctacagaagattgttttcctaccatccgcctacctcatggatctgtggagatggaacgtgttcagtggtgacatcacaagtgacagttacaactgtgaatggtggaaacttagggagaaatatcaaggtattgaaccaccagtggacagaagcgaagatgattttgatgctggagcaaaataccacatcatagccagtgttccatacattaggtactttgtaagctttgtcatycartttcagttctacagagccgc

SEQ ID NO: 13 - protein, 368 aa, Blattella germanica
TKSLDHEELKHVWVEWRKVSGEKCRGLFEHYIALSNEAATLNNFTNNAEFWLRDYEAPDFKEQVSKLWEKIEPLYLQIHAYVRRKLRETYGEELVKPRGPIPAHLLGNMWSQTWNSILDISTPFPGKEAIDVTPQMVKQGYTPLKMFKLSEQFFTSLNLSAMPEMFWEKSILQKPSDRELVCHASAWDFYDGKDFRIKQCTRVNMDDLFTAHHEMGHIQYFLQYKDQPTVYKEGANPGFHEAVGDVMALSVSTPKHLRKIGLLEESDKGEDYEATINYLFLQGLQKIVFLPSAYLMDLWRWNVFSGDITSDSYNCEWWKLREKYQGIEPPVDRSEDDFDAGAKYHIIASVPYIRYFVSFVIQFQFYRA

SEQ ID NO: 14 - DNA, 736 bp, Blattella germanica
  CDS (9)..(734)
gcccttagagcctggacccggaagagctgaaacatgtgtgggtggaatggaggaaggtctctggagagaaatgcagaggactttttgagcattatatcgcactcagcaatgaggctgccgcacttaacaatttcaccaacaacgctgaattttggttaagagattatgaagcacctgatttcaaagagcaagtatctaaactttgggaaaaaatagaaccactatacttgcagattcatgcatatgtcaggaggaagctaagagagacgtatggagaggagctcgtgaaaccacgtggaccaataccagcacatcttttaggtaacatgtggtctcagaagtggggtaacattctgaacattacaattccttatcctggtagacctcgagtagatgtgaccaatgcaatggtacaacagggatacactcctttgaagatgttcaaactatcagaacaattcttcacatctctcaaccttagtgcaatgcccgaaatgttctgggagaagtcaattttgcagaarccttcagatagagaacttgtttgtcatgcttctgcatgggacttctatgacggaaaagatttcaggatcaaacaatgtacgagggttaacatggatgatcttttcactgcacatcatgagatgggccacatccagtatttccttcaatataaagatcagcccacagtctacaaggaaggtgctaatccagggttccacgaggctgtcggtga

SEQ ID NO: 15 - protein, 242 aa, Blattella germanica
SLDPEELKHVWVEWRKVSGEKCRGLFEHYIALSNEAAALNNFTNNAEFWLRDYEAPDFKEQVSKLWEKIEPLYLQIHAYVRRKLRETYGEELVKPRGPIPAHLLGNMWSQKWGNILNITIPYPGRPRVDVTNAMVQQGYTPLKMFKLSEQFFTSLNLSAMPEMFWEKSILQKPSDRELVCHASAWDFYDGKDFRIKQCTRVNMDDLFTAHHEMGHIQYFLQYKDQPTVYKEGANPGFHEAVG

SEQ ID NO: 16 - DNA, 736 bp, Blattella germanica
  CDS (3)..(734)
  misc_feature (18)..(20): The codon “smr” codes for Glu, Ala, Gln or Pro
tcaccaagagcctggacsmrgargarytgaaacatgtgtgggtggaatggaggaaggtctctggagagaaatgcagaggactttttgagcattatatcgcactcagcaatgaggctgccacacttaacaatttcaccaacaacgctgaattttggttaagagattatgaagcacctgatttcaaagagcaagtatctaaactttgggaaaaaatagaaccactatacttgcagattcatgcatatgtcaggaggaagctaagagagacgtatggagaggagctcgtgaaaccacgtggaccaataccagcacatcttttaggaaatatgtggtcccagacatggaatagtattctggatatttcaacacccttccctggaaaagaagcaatagatgtcacccctcaaatggtcaaacagggatacactcctttgaagatgttcaaactatcagaacaattcttcacatctctcaaccttagtgcaatgcccgaaatgttctgggagaagtcaattttgcagaaaccttcagatagagaacttgtttgtcatgcttctgcatgggacttctatgacggaaaagatttcaggatcaaacagtgcactagcattacaatgcatgaccttttcactgttcatcatgaaatgggccatatccaatatttcatacaatatagtcatcaacctacagtatacaaagagggttccaatccagggtttcacgaggctgtcggtga

SEQ ID NO: 17 - protein, 244 aa, Blattella germanica
  MOD_RES (6)..(6): Glu, Ala, Gln or Pro
TKSLDXEELKHVWVEWRKVSGEKCRGLFEHYIALSNEAATLNNFTNNAEFWLRDYEAPDFKEQVSKLWEKIEPLYLQIHAYVRRKLRETYGEELVKPRGPIPAHLLGNMWSQTWNSILDISTPFPGKEAIDVTPQMVKQGYTPLKMFKLSEQFFTSLNLSAMPEMFWEKSILQKPSDRELVCHASAWDFYDGKDFRIKQCTSITMHDLFTVHHEMGHIQYFIQYSHQPTVYKEGSNPGFHEAVG

SEQ ID NO: 18 - DNA, 1361 bp, Blattella germanica
  CDS (3)..(1361)
  misc_feature (3)..(5): The codon “wag” codes for Lys or is a stop codon
cywagtgcgatttggctttrgaaccwgagttgacggttgtcatgggaaggagcagggactgggatgagttgcagcatcagtggctggaatggaggcgattgacgggccagaaaatccgcgatttgttcgagcaagttgtggaccttaccaacaaggccgcgcaattgaatcattttaaggatggcgcagagtactggatgtttccttacgaatctccgaatttccgatatgaattggacgaagtttgggaagaagtccggccattgtatgaactactccacgcctatgtacggaggaagctaagggacctttatggtcctgataagctgagtcgaaaggcgcctttaccagcacacatacttggtgacatgtggggacaatcctggagtaacattctggatgttaccattccgtatccaggaaagaatttcttagatgtgacagaagaaatgrtcaaccaggggtacactccattgacaatgttccaactggcggaagaattctttttgtctctgaatatgagtgtgttgcctcctgaattctgggcwggctccataattgaagaattrcctggaagggctatcatatgtcaaccttcagcctgggatttytgcaacagacgtgactacaggataaaaatgtgtactcaggtgaacatgaaggacttygtaacagttcaccacgaagtcgcacatctgcaatattttctgcagtatcgccatctacccaaggtctttcgagatggagctaatcctggkttccacgaggcwgtyggwgaggctgtggcattgtcaatatcgacgccnaggcatttgcaggacctcggtctggtccaagggtcagttgatgacacccctcacaacataaacatgctttatgcactcgctttagacaaactgcctttccttggcttcagcctggccctggaccaatggcggtgggacattttcgaaggctctgtgtcacaagatcaatacaattgccattggtggaggcttagagagaagtattctggagtgaaaccacctgtgctgaggtcagaagtagacttcgatcctgggtccaagtaccatgtcgctgccaatatgccctacattcggtacttcgtgggtacagtacttcagtttcagctgcatagtgccttatgcaaagcagctgggcagtttgaccctgatcaacccctgacgaagccgctctacaaatgcgacatctaccgcagcaaagaggccggaaaactactcaaggaaatgatgagattaggctcatcgctaccttggactgaggtcctgtatgcagctactggagaaaccaggctcaatggacgacacatcagggaatacttcaagcccatgcacgattgg

SEQ ID NO: 19 - protein, 453 aa, Blattella germanica
  MOD_RES (1)..(1): May or may not be present
KCDLALEPELTVVMGRSRDWDELQHQWLEWRRLTGQKIRDLFEQVVDLTNKAAQLNHFKDGAEYWMFPYESPNFRYELDEVWEEVRPLYELLHAYVRRKLRDLYGPDKLSRKAPLPAHILGDMWGQSWSNILDVTIPYPGKNFLDVTEEMXNQGYTPLTMFQLAEEFFLSLNMSVLPPEFWAGSIIEELPGRAIICQPSAWDFCNRRDYRIKMCTQVNMKDFVTVHHEVAHLQYFLQYRHLPKVFRDGANPGFHEAVGEAVALSISTPRHLQDLGLVQGSVDDTPHNINMLYALALDKLPFLGFSLALDQWRWDIFEGSVSQDQYNCHWWRLREKYSGVKPPVLRSEVDFDPGSKYHVAANMPYIRYFVGTVLQFQLHSALCKAAGQFDPDQPLTKPLYKCDIYRSKEAGKLLKEMMRLGSSLPWTEVLYAATGETRLNGRHIREYFKPMHDW

SEQ ID NO: 20 - DNA, 387 bp, Blattella germanica
  CDS (3)..(386)
ccaagtgcgatttggctctagatcctgacttggctcgaattatggcacattcaagagattatgatgaacaattacatgtatggcttgcatggcgagatgctattggacctcaaatacgagataaatatattcagtatgttcagatggccaatcatgctgcaaggttgaatggttttcatgatgctggacagcaacaaagagaagcatatgaagattctgatatcaattcacaacttactgaactgtgggcaacattagccccactctacagagagttgcatgcatatgtaagacgccatcttgtccagaggtatggaccagaaagagtgagacctgatggaccaatgccagcacatcttttaggaaatatgtggtcaagggcgaatt

SEQ ID NO: 21 - protein, 128 aa, Blattella germanica
KCDLALDPDLARIMAHSRDYDEQLHVWLAWRDAIGPQIRDKYIQYVQMANHAARLNGFHDAGQQQREAYEDSDINSQLTELWATLAPLYRELHAYVRRHLVQRYGPERVRPDGPMPAHLLGNMWSRAN

SEQ ID NO: 22 - protein, 6 aa, Artificial Sequence
  Description of Artificial Sequence Synthetic 6xHis tag
HHHHHH

(19) United States
(12) Patent Application Publication
(10) Pub. No.: US 2012/0258929 A1
(43) Pub. Date: Oct. 11, 2012

(54) AGENT THAT MODULATES PHYSIOLOGICAL CONDITION OF PESTS, INVOLVED IN INSECT PEPTIDYL-DIPEPTIDASE A ACTIVITY

(75) Inventors: Yasutaka Shimokawatoko, Kobe-shi (JP); Marc Van De Craen, Aalter (BE); Irene Nooren, Oegstgeest (NL); Sandra Turconi, Destelbergen (BE); Yann Naudet, Schepdaal (BE); Guy Nys, Aalter (BE); Jurgen Debaveye, Gent-Ledeberg (BE)

(73) Assignee: SUMITOMO CHEMICAL COMPANY, LIMITED, Chuo-ku, Tokyo (JP)

(21) Appl. No.: 11/993,586

(22) PCT Filed: Jun. 23, 2006
(86) PCT No.: PCT/JP06/13039

§ 371 Date: Aug. 31, 2009

(30) Foreign Application Priority Data
Jun. 23, 2005 (JP) 2005-183031

(51) Int. Cl. A01N 43/08; A01N 43/36; C07D 285/135; A01N 43/824; C07D 225/06; A01N 43/46; C07J 1/00; A01N 45/00; C07D 209/52; A01N 43/38; C07D 217/26; A01N 43/42; C07H 19/067; C07D 277/54; A01N 43/78; A01P 7/04; C12N 15/57; C12Q 1/37; C12N 9/48; C12Q 1/68; C12N 15/66; C12N 5/10; C12N 15/64; C12N 7/01; C07D 207/16

(52) U.S. Cl. 514/50; 548/533; 514/423; 548/140; 514/363; 540/523; 514/212.07; 552/636; 514/178; 548/452; 514/412; 546/147; 514/307; 536/28.53; 548/184; 514/369; 552/528; 536/23.2; 435/24; 435/212; 435/6.12; 435/320.1; 435/348; 435/455; 435/235.1

(57) ABSTRACT

The present invention provides an agent that modulates physiological condition of pests, wherein the agent has an ability to modulate the activity of an insect peptidyl-dipeptidase A; a method for assaying pesticidal activity of a test substance, which comprises a step of measuring the activity of a peptidyl-dipeptidase A in a reaction system in which the peptidyl-dipeptidase A contacts with a test substance, and the like.

### TECHNICAL FIELD

The present invention relates to an agent that modulates physiological condition of pests, involved in insect peptidyl-dipeptidase A activity, and the like.

### BACKGROUND ART

Regarding insect peptidyl-dipeptidase A, for example, it has been reported:

that in Drosophila peptidyl-dipeptidase A, there are the following two enzymes Ance: Angiotensin converting enzyme and Acer: Angiotensin-converting enzyme-related enzyme and, in D. melanogaster larva, the high level of peptidyl-dipeptidase A activity has been detected in the midgut, the brain and the haemolymph (for example, Houard et al., Eur J Biochem., 257(3):599-606, 1998);

that Ance and Acer mRNAs, evidenced by Northern assays, are detected during embryogenesis and throughout all post-embryonic stages of development (for example, Tatei et al., Mech Dev., 51(2-3):157-68, 1995; Taylor et al., Gene., 181(1-2), 191-7, 1996 etc.);

that deletion mutants for these two Drosophila melanogaster peptidyl-dipeptidase A genes (Ance and Acer) showed recessive lethal phenotypes at early larval stages (for example, Tatei et al., Mech Dev., 51(2-3):157-68, 1995 etc.);

that Ance plays a role in contractions of the heart, gut or testes (for example, Tatei et al., Mech Dev., 51(2-3):157-68, 1995 etc.);

that in Locusta migratoria, a possible role for Peptidyl-dipeptidase A in the metabolism of locustamyotropins-like neuropeptides, is suggested (for example, Isaac et al., Biochem J.; 330 (Pt 1):61-5, 1998; Isaac et al., Ann NY Acad Sci, 839:288-292, 1998 etc.); and

that peptidyl-dipeptidase A has been evidenced in Anopheles stephensi female mosquitoes where the enzyme is induced by a blood meal (for example, Ekbote et al., FEBS Lett; 455(3):219-22, 1999), and peptidyl-dipeptidase A accumulates in developing ovaries and passes into the mosquito eggs, where it may play a role in the metabolism of peptides during embryogenesis.

Discovery of agricultural chemicals has traditionally been based on a random screening process, often directly testing the effects of specific chemicals on whole organisms, such as insects, fungi and/or plants and determining biological activity. Once chemical compounds with the appropriate biological activity are discovered, more intense research is required to specifically determine the mode of action or site of action of these compounds at the molecular level, in order to predict safety and environmental load of these compounds.

### DISCLOSURE OF INVENTION

This invention describes a more target-based approach of screening agricultural chemicals, whereby compounds are screened against a specific target that has been identified as biologically and/or physiologically relevant with intent of chemically interfering with the target site to control insects or other pest organisms.

Specifically, this invention describes that an agent that modulates physiological condition of pests and having an ability to modulate the activity of an insect peptidyl-dipeptidase A is useful to control pests.

That is, the present invention provides:

1. An agent that modulates physiological condition of pests,

wherein the agent has an ability to modulate the activity of an insect peptidyl-dipeptidase A;

2. An agent according to item 1, wherein the peptidyl-dipeptidase A is a cotton aphid peptidyl-dipeptidase A;

3. An agent according to item 1, wherein the agent is a pesticidal agent;

4. An agent according to item 1, wherein the ability to modulate the activity of an insect peptidyl-dipeptidase A is an ability to inhibit a reaction of the insect peptidyl-dipeptidase A with o-aminobenzoylglycyl-p-nitro-L-phenylalanyl-L-proline;

5. A pesticidal agent which comprises a substance that has an ability to modulate the activity of an insect peptidyl-dipeptidase A or an agriculturally acceptable salt of the substance as an active ingredient;

6. A pesticidal agent according to item 5, wherein the substance has an ability to inhibit a reaction of the insect peptidyl-dipeptidase A with o-aminobenzoylglycyl-p-nitro-L-phenylalanyl-L-proline;

7. A pesticidal agent according to item 6, wherein the substance has an ability to inhibit the reaction of the insect peptidyl-dipeptidase A with o-aminobenzoylglycyl-p-nitro-L-phenylalanyl-L-proline in a cell-free system, wherein in the presence of the substance of 10 or more the activity of the peptidyl-dipeptidase A is lower than that in the absence of the substance;

8. A pesticidal agent according to item 6, wherein the substance has an ability to inhibit a reaction of the insect peptidyl-dipeptidase A with o-aminobenzoylglycyl-p-nitro-L-phenylalanyl-L-proline in a cell-free system with an IC50 of 100 μM or less;

9. A method for assaying pesticidal activity of a test substance, which comprises:

(1) a first step of measuring the activity of a peptidyl-dipeptidase A selected from the following group A in a reaction system in which the peptidyl-dipeptidase A contacts with a test substance; and

(2) a second step, of evaluating the pesticidal activity of the test substance based on the difference obtained by comparing the activity measured in the first step with the activity of a control.

### <Group A>

(a) a protein comprising the amino acid sequence of SEQ ID NO: 1;

(b) a protein comprising an amino acid sequence with deletion, addition or substitution of one or more amino acids in the amino acid sequence of SEQ ID NO: 1, wherein the protein has peptidyl-dipeptidase A activity;

(c) a protein comprising an amino acid sequence that has sequence identity of 65% or more to the amino acid sequence of SEQ ID NO: 1, wherein the protein has peptidyl-dipeptidase A activity;

(d) a protein comprising the amino acid sequence encoded by the nucleotide sequence of SEQ ID NO: 2;

(e) a protein comprising an amino acid sequence encoded by a nucleotide sequence that has sequence identity of 65% or more to the nucleotide sequence of SEQ ID NO: 2, wherein the protein has peptidyl-dipeptidase A activity;

(f) a protein comprising an amino acid sequence encoded by a polynucleotide, wherein the polynucleotide hybridizes under a stringent condition to a polynucleotide comprising a nucleotide sequence complementary to the nucleotide sequence of SEQ ID NO: 2, and wherein the protein has peptidyl-dipeptidase A activity;

(g) a protein comprising an amino acid sequence of an insect peptidyl-dipeptidase A; and

(h) a protein comprising an amino acid sequence of a cotton aphid peptidyl-dipeptidase A;

10. A method for screening a pesticidal substance, which comprises selecting a substance having the pesticidal activity that is evaluated by the method according to item 9;

11. A pesticidal agent which comprises a substance selected by the method according to item 10 or agriculturally acceptable salts thereof as an active ingredient;

12. A method for controlling pests which comprises applying an effective amount of the pesticidal agent according to item 5, 6, 7, 8 or 11 to the pest, habitat of the pest or plant to be protected from the pest;

13. A method for controlling pests which comprises:

identifying a substance having the pesticidal activity that is evaluated by the method according to item 9, and

contacting the pest with the identified pesticidal substance;

14. An insect peptidyl-dipeptidase A comprising an amino acid sequence selected from the following group B:

### <Group B>

(a) the amino acid sequence of SEQ ID NO: 1;

(b) an amino acid sequence with deletion, addition or substitution of one or more amino acids in the amino acid sequence of SEQ ID NO: 1, wherein the amino acid sequence has peptidyl-dipeptidase A activity;

(c) an amino acid sequence that has sequence identity of 65% or more to the amino acid sequence of SEQ ID NO: 1, wherein the amino acid sequence has peptidyl-dipeptidase A activity;

(d) the amino acid sequence encoded by the nucleotide sequence of SEQ ID NO: 2;

(e) an amino acid sequence encoded by a nucleotide sequence that has sequence identity of 65% or more to the nucleotide sequence of SEQ ID NO: 2, wherein the amino acid sequence has peptidyl-dipeptidase A activity;

(f) an amino acid sequence encoded by a polynucleotide, wherein the polynucleotide hybridizes under a stringent condition to a polynucleotide comprising a nucleotide sequence complementary to the nucleotide sequence of SEQ ID NO: 2, wherein the amino acid sequence has peptidyl-dipeptidase A activity; and

(g) an amino acid sequence of a cotton aphid peptidyl-dipeptidase A;

15. Use of an insect peptidyl-dipeptidase A as a reagent that provides an indicator to evaluate pesticidal activity;

16. Use of an insect peptidyl-dipeptidase A according to item 14 as a reagent that provides an indicator to evaluate pesticidal activity;

17. A polynucleotide which comprises a nucleotide sequence encoding an amino acid sequence of a peptidyl-dipeptidase A according to item 14;

18. A polynucleotide according to item 17, which comprises the nucleotide sequence of SEQ ID NO: 2;

19. A polynucleotide which comprises a nucleotide sequence complementary to a nucleotide sequence of a polynucleotide according to item 17 or 18;

20. A polynucleotide which comprises:

a partial nucleotide sequence of a polynucleotide according to item 17 or 18; or

a nucleotide sequence complementary to the partial nucleotide sequence;

21. A polynucleotide according to item 20, which comprises a nucleotide sequence of SEQ ID NO: 3 or 4;

22. A method for obtaining a polynucleotide comprising a nucleotide sequence encoding an amino acid sequence of a peptidyl-dipeptidase A, which comprises:

a step of amplifying a desired polynucleotide by polymerase chain reaction using as a primer a polynucleotide according to item 20 or 21;

a step of identifying the desired polynucleotide amplified; and

a step of recovering the identified polynucleotide;

23. A method for obtaining a polynucleotide comprising a nucleotide sequence encoding an amino acid sequence of a peptidyl-dipeptidase A, which comprises:

a step of detecting a desired polynucleotide by hybridization using as a probe a polynucleotide according to item 19, 20 or 21;

a step of identifying the desired polynucleotide detected; and

a step of recovering the identified polynucleotide;

24. A circular polynucleotide comprising a nucleotide sequence of a polynucleotide according to item 17 or 18, wherein the nucleotide sequence is operably linked to a baculovirus promoter;

25. A circular polynucleotide according to item 24, wherein the promoter is a polyhedrin gene promoter;

26. A circular polynucleotide according to item 24 or 25, wherein the polynucleotide comprises a replication origin for autonomous replication in a host cell;

27. A circular polynucleotide according to item 24, 25 or 26, wherein the polynucleotide comprises a nucleotide sequence of a baculovirus shuttle vector and is capable of propagating as a virus in an insect cell;

28. A method for producing a circular polynucleotide, which comprises ligating a polynucleotide according to item 17 or 18 into a vector;

29. A transformant in which a polynucleotide according to item 17 or 18 is introduced;

30. A transformant according to item 29, wherein the transformant is a transformed insect cell;

31. A method for producing a transformant, which comprises introducing a polynucleotide according to item 17 or 18 into a host cell;

32. A recombinant baculovirus comprising within its genome a polynucleotide according to item 17 or 18;

33. A method for producing a peptidyl-dipeptidase A, which comprises a step of culturing the transformant according to item 29 or 30 and recovering a produced peptidyl-dipeptidase A;

34. Use of a peptidyl-dipeptidase A according to item 14 or a polynucleotide according to any one of items 17 to 21 as a research tool;

35. Use according to item 34, wherein the research tool is an experimental tool for screening a pesticidal substance; and

36. A system which comprises:

a means to input, store and manage a data information of an ability of test substances, wherein the ability is an ability to modulate the activity of an insect peptidyl-dipeptidase A;

a means to query and retrieve the data information based on a desired criterion; and
a means to display and output the result which is queried and retrieved.

### MODES FOR CARRYING OUT THE INVENTION

The present invention will be explained in detail below.

In the present invention, the “pests” indicates small animals which cause harm or discomfort to life of the people by harming man and animals directly or by damaging crops. Examples thereof include arthropod such as insects, mites and ticks and Nematoda, and typical examples of which are as follows:

### Hemiptera:

Delphacidae such as Laodelphax striatellus, Nilaparvata lugens and Sogatella furcifera, Deltocephalidae such as Nephotettix cincticeps and Empoasca onukii, Aphididae such as Aphis gossypii and Myzus persicae, Pentatomidae, Aleyrodidae such as Trialeurodes vaporariorum, Bemisia tabaci and Bemisia argentifolli, Coccidae, Tingidae, Psyllidae, etc.

### Lepidoptera:

Pyralidae such as Chilo suppressalis, Cnaphalocrocis medinalis, Ostrinia nubilalis and Parapediasia teterrella, Noctuidae such as Spodoptera litura, Spodoptera exigua, Pseudaletia separata, Mamestra brassicae, Agrotis ipsilon, Trichoplusia spp., Heliothis spp., Helicoverpa spp. and Earias spp., Pieridae such as Pieris rapae crucivora, Tortricidae such as Adoxophyes orana fasciata, Grapholita molesta and Cydia pomonella, Carposinidae such as Carposina niponensis, Bucculatricidae such as Lyonetia clerkella, Gracillariidae such as Phyllonorycter ringoniella, Phyllocnistidae such as Phyllocnistis citrella, Yponomeutidae such as Plutella xylostella, Gelechiidae such as Pectinophora gossypiella, Arctiidae, Tineidae, etc.

### Diptera:

Culex such as Culex pipiens pallens, Cules tritaeniorhynchus and Culex quinquefasciatus, Aedes such as Aedes aegypti and Aedes albopictus, Anopheles such as Anophelinae sinensis, Chironomidae, Muscidae such as Musca domestica and Muscina stabulans, Calliphoridae, Sarcophagidae, Fannia canicularis, Anthomyiidae such as Delia Platura and Delia antigua, Trypetidae, Drosophilidae, Psychodidae, Simuliidae, Tabanidae, Stomoxyidae, Agromyzidae, etc.

### Coleoptera:

Diabrotica such as Diabrotica virgifera virgifera and Diabrotica undecimpunctata howardi, Scarabaeidae such as Anomala cuprea and Anomala rufocuprea, Curculionidae such as Sitophilus zeamais, Lissorphoptrus oryzophilus and Calosobruchys chinensis, Tenebrionidae such as Tenebrio molitor and Tribolium castaneum, Chrysomelidae such as Oulema oryzae, Aulacophora femoralis, Phyllotreta striolata and Leptinotarsa decemlineata, Anobiidae, Epilachna spp. such as Epilachna vigintioctopunctata, Lyctidae, Bostrychidae, Cerambycidae, Paederus fuiscipes, etc.

### Thysanoptera:

Thripidae such as Thrips spp. including Thrips palmi, Frankliniella spp. including Frankliniella occidentalis and Sciltothrips spp. including Sciltothrips dorsalis, Phlaeothripidae, etc.

### Hymenoptera:

### Tenthredinidae, Formicidae, Vespidae, etc.

### Dictyoptera:

### Blattidae, Blattellidae, etc.

### Orthoptera:

### Acrididae, Gryllotalpidae etc.

### Siphonaptera:

Pulex irritans, etc.

### Anoplura:

Pediculus humanus capitis, etc.

### Isoptera:

### Termitidae, etc.

### Acarina:

Tetranychidae such as Tetranychus urticae, Tetranychus kanzawai, Panonychus citri, Panonychus ulmi, and Oligonychus spp., Eriophyidae such as Aculops pelekassi and Aculus schlechtendali, Tarsonemidae such as Polyphagotarsonemus latus, Tenuipalpidae, Tuckerellidae, Ixodidae such as Haemaphysalis longicornis, Haemaphysalisflava, Dermacentortaiwanicus, Ixodes ovatus, Ixodes persulcatus and Boophilus microplus, Acaridae such as Tyrophagus putrescentiae, Dermanyssidae, Cheyletidae such as Dermatophagoides farinae and Dermatophagoides ptrenyssnus, such as Cheyletus eruditus, Cheyletus malaccensis and Cheyletus moorei, Dermanyssus spp., etc.

### Nematodes:

Pratylenchus coffeae, Pratylenchus fallax, Heterodera glycines, Globodera rostochiensis, Meloidogynehapla, Meloidogyne incognita, etc.

In the present invention, the “modulate physiological condition of pests” indicates changing condition such as various phenomena in a living body which are maintained for living in pests, for example, function such as aspiration, digestion, secretion, body liquid circulation, metabolism, neurotransmission and the like, or mechanism thereof into condition apart from usual condition. Examples include changing condition by cessation of aspiration so that oxygen necessary for internal metabolism of pests is not supplied, and changing condition by cessation of function of neurotransmission of pests so that various movements of pests are ceased.

In the present invention, the “agent which modulates physiological condition of pests” is an agent which can modulate physiological condition of pests when being applied to pests.

In the present invention, the “insect peptidyl-dipeptidase A” indicates a peptidyl-dipeptidase A that occurs in insect, among peptidyl-dipeptidase A present in various organisms. Herein, insect is an animal classified under Animalia, Arthropoda, Insecta, and examples of which include arthropod of the order Protura, Collembola, Diplura, Thysanura, Ephemeroptera, Odonata, Plecoptera, Grylloblattodea, Orthoptera, Phasmatodea, Dermaptera, Mantodea, Blattaria, Isoptera, Embioptera, Psocoptera, Mallophaga, Anoplura, Thysanoptera, Hemiptera, Neuroptera, Mecoptera, Trichoptera, Lepidoptera, Coleoptera, Diptera, Hymenoptera, Siphonaptera, Strepsiptera, and the like.

Peptidyl-dipeptidase A (EC 3.4.15.1; also referred to as angiotensin I-converting enzyme (ACE), kininase II or dipeptidyl carboxypeptidase I) is a metallopeptidases that hydrolytically cleaves carboxy-terminal dipeptides from short peptide.

Peptidyl-dipeptidase A has the activity to cleave dipeptides from the carboxyl terminus of short peptide hormones. Although an endogenous substrate of peptidyl-dipeptidase A in insect is not identified, activity of peptidyl-dipeptidase A can be measured using a synthetic substrate, o-aminobenzoylglycyl-p-nitrophenylalanylproline (Abz-Gly-Phe (NO₂)-Pro) (manufactured by Bachem, catalogue No. M-1100). This substrate is an internally quenched peptide that shows very weak fluorescence. This quenching is abolished upon cleavage of the peptide-bond Gly-Phe(NO₂) and the increasing fluorescence is proportional to the peptidyl-dipeptidase A activity. More specifically, the activity of peptidyl-dipeptidase A can be measured according to the method described in Carmel et al. (1979) Clinica Chimica Acta, 93, 215-220.

The activity of peptidyl-dipeptidase A can be measured using in vitro assays using natural or synthetic substrates of the enzyme. For instance, the peptidyl-dipeptidase A activity can be measured using a radioactivity-based assay with radiolabeled angiotensin I substrate as described by Huggins and Thampi, Life Sci. 7(12), 1968, 633-639. Another example to monitor the peptidyl-dipeptidase A activity using angiotensin I as substrate is based on reversed-phase HPLC separation of the product formed, as described by Meng et al, Biochem Pharmacol 50, 1995, 1445-1450. Similarly, numerous assays exist which can be used to determine the peptidyl-dipeptidase A activity using synthetic substrates instead of the natural substrate such as angiotensin I and bradykinin. These methods include measuring the products of the cleavage of radiolabeled substrates, fluorometric or colorimetric analyses of cleaved substrates. Types of such assays are described by Holmquist et al., Analytical Biochemistry 95, 540-548 (1979); Carmel and Yaron, Eur J Biochem 87, 265-273 (1978); Araujo et al, Biochemistry 39(29), 8519-8525 (2000); Shepley et al, Journal of Pharmaceutical and Biomedical Analysis, 6(3), 241-251 (1988); Cushman and Cheung, Biochem Pharmacol. 20(7):1637-1648 (1971); Groff et al, Clin Chem 39(3), 400-404 (1993); Dive et al, PNAS 96, 4330-4335 (1999); Cheviron et al, Analytical Biochemistry 280, 58-64 (2000); Meng and Oparil, Journal of Chromatography A 743, 105-122 (1996).

Among the aforementioned various methods of measuring activity of a peptidyl-dipeptidase A, a reaction using the o-aminobenzoylglycyl-p-nitro-L-phenylalanyl-L-proline as a substrate is preferable for automatic and effective measurement of the peptidyl-dipeptidase A activity of many samples. Specific examples include the method described in the already-reported article (Carmel et al. (1979) Clinica Chimica Acta, 93, 215-220).

In addition, a method of measuring activity of an insect peptidyl-dipeptidase A can be performed by a similar method to that described above.

Several amino acid sequences of peptidyl-dipeptidase A have been identified in different insect species, for example in D. melanogaster ANCE (accession No. NP_{—}477046), in Haematobia irritans (accession No. S65472), in Anopheles gambiae (accession No. XP_{—}318838), in Bombyx mori (accession No. BAA97657), in Apis mellifera (accession No. XP_{—}393561), in Locusta migratoria (accession No. AAR85358), and the like, which can be found in public databases. Also, several nucleotide sequences of peptidyl-dipeptidase A genes have been identified in different insect species, for example in D. melanogaster ANCE (accession No. NM_{—}057698), in Haematobia irritans (accession No. L43965), in Anopheles gambiae (accession No. AAAB01008980), in Bombyx mori (accession No. AB026110), in Apis mellifera (accession No. XM_{—}393561), in Locusta migratoria (accession No. AY487174), and the like, which can be found in public databases.

In addition, according to the methods described below, an amino acid sequence of peptidyl-dipeptidase A and a nucleotide sequence of peptidyl-dipeptidase A gene can be identified from a cotton aphid. The identified amino acid sequence of cotton aphid peptidyl-dipeptidase A is shown in SEQ ID NO: 1, and the nucleotide sequence of cotton aphid peptidyl-dipeptidase A gene is shown in SEQ ID NO: 2.

Several amino acid sequences of peptidyl-dipeptidase A have been identified in animals other than insect, for example in Homo sapiens (accession No. NP_{—}000780), in Bos taurus (accession No. 1919242A), in Ceanorhabditis elegans (accession No. AAA98719), which can be found in public databases. Also, several nucleotide sequences of peptidyl-dipeptidase A genes have been identified in animals other than insect, for example in Homo sapiens (accession No. NM_{—}000789), in Bos taurus (accession No. AJ309016), in Ceanorhabditis elegans (accession No. U56966), which can be found in public databases.

Table 1 shows Sequence identity of the amino acid sequence of cotton aphid peptidyl-dipeptidase A (SEQ ID NO: 1) and the nucleotide sequence of cotton aphid peptidyl-dipeptidase A gene (SEQ ID NO: 2) with the sequence of peptidyl-dipeptidase A and gene thereof found in other animals.

TABLE 1

| | Identity of | Identity of |
|---|---|---|
| | amino acid | nucleotide |
| | sequence (%) vs | sequence (%) vs |
| Origin of sequence | SEQ ID NO: 1 | SEQ ID NO: 2 |
| D. melanogaster ANCE | 37.8 | 45.5 |
| Haematobia irritans | 35.1 | 49.4 |
| Anopheles gambiae | 34.4 | 49.4 |
| Bombyx mori | 51.5 | 53.7 |
| Apis mellifera | 61.9 | 63.3 |
| Locusta migratoria | 51.3 | 48.6 |
| Homo sapiens | 31.3 | 47.8 |
| Bos taurus | 31.1 | 53.8 |
| Ceanorhabditis elegans | 29.3 | 44.7 |

An ability to modulate the activity of an insect peptidyl-dipeptidase A refers to an ability to increase or decrease activity of an insect peptidyl-dipeptidase A, that is, means an ability to activate a peptidyl-dipeptidase A, or an ability to inhibit activity of a peptidyl-dipeptidase A. And, a test substance can be added to the reaction system for measuring peptidyl-dipeptidase A activity to investigate influence of the test substance on the peptidyl-dipeptidase A activity.

As a substance having an ability to activate a peptidyl-dipeptidase A, for example, a chloride ion (Cl⁻) is known (Lamango N S et al., Biochem J. 1996 Mar. 1; 314 (Pt 2):639-46.) and, as a substance having an ability to inhibit the activity of a peptidyl-dipeptidase A, benazepril, quinapril, captopril, enalapril and the like are known.

However, there has never been finding or has never been known suggestion that these substances modulate physiological condition of pests, particularly, can be an active ingredient of a pesticidal agent.

An IC₅₀ value of a test substance in the reaction means a concentration of a test substance at which 50% of the activity of the reaction with no test substance is inhibited. The IC₅₀ value of a test substance can be determined by adding test substances of different concentrations to the peptidyl-dipeptidase A activity measuring reaction system, measuring the peptidyl-dipeptidase A activity (response) at each concentration of added test substance (dose), producing a dose-response curve, and calculating a concentration of the added test substance, at which the peptidyl-dipeptidase A activity is 50% inhibited. More specifically, a dose-response curve may be produced using 4 Parameter Logistic Model or Sigmoidal Dose-Response Model:

$$f ( x ) = ( A + ( ( B - A ) / ( 1 + ( ( C / x ) \hat{} D ) ) ) \\ f ( x ) = A + \frac{B - A}{1 + {( C / x )}^{D}}$$

to calculate the IC₅₀. Practically, the IC₅₀ value may be calculated using XLfit (manufactured by IDBS) which is a commercially available calculating software.

An agent that has an ability to modulate the activity of an insect peptidyl-dipeptidase A is an agent containing as an active ingredient a substance having an ability to modulate the activity of an insect peptidyl-dipeptidase A.

In the present invention, the “agent that modulates physiological condition of pests, wherein the agent has an ability to modulate the activity of an insect peptidyl-dipeptidase A” is an agent having an ability to modulate the activity of insect peptidyl-dipeptidase A specified by the aforementioned measuring method, and means an agent that can modulate physiological condition of pests. Preferable examples of the agent include an agent in which an insect peptidyl-dipeptidase A is a cotton aphid peptidyl-dipeptidase A. In addition, preferable examples of the agent include an agent in which an agent that modulates physiological condition of pests is a pesticidal agent. In addition, preferable examples of the agent include an agent in which an ability to modulate the activity of an insect peptidyl-dipeptidase A is an ability to inhibit a reaction using the o-aminobenzoylglycyl-p-nitro-L-phenylalanyl-L-proline as a substrate.

In the present invention, the “pesticidal agent” indicates an agent having an ability to control the pests.

Examples of a method for measuring an ability to control pests include, in addition to the methods disclosed in the present invention, a method of measuring pesticidal activity on the pests. Specifically, for example, the pesticidal activity can be measured according to the following method.

According to the method described in Handbook of Insect Rearing Vol. 1 (Elsevier Science Publishers 1985), pp. 35 to pp. 36 except that a sterilized artificial feed having the following composition (Table 2) is prepared, and a solution of a test agent in DMSO is added at 0.5% by volume of the artificial feed and is mixed, a cotton aphid is reared, the number of surviving cotton aphids is investigated after 6 days, and a controlling value is obtained according to the following equation.

TABLE 2

| | (mg/100 ml) |
|---|---|
| Amino acid | |
| L-Alanine | 100.0 |
| L-arginine | 275.0 |
| L-Asparagine | 550.0 |
| L-Aspartic acid | 140.0 |
| L-cysteine | 40.0 |
| (hydrochloride) | |
| L-glutamic acid | 140.0 |
| L-glutamine | 150.0 |
| L-glycine | 80.0 |
| L-histidine | 80.0 |
| L-isoleucine | 80.0 |
| L-leucine | 80.0 |
| L-lysine | 120.0 |
| (hydrochloride) | |
| L-methionine | 80.0 |
| L-phenylalanine | 40.0 |
| L-proline | 80.0 |
| L-serine | 80.0 |
| L-threonine | 140.0 |
| L-tryptophan | 80.0 |
| L-tyrosine | 40.0 |
| L-valine | 80.0 |
| Vitamins | |
| Ascorbic acid | 100.0 |
| Biotin | 0.1 |
| Calcium | 5.0 |
| pantothenate | |
| Choline | 50.0 |
| chloride | |
| Inositol | 50.0 |
| Nicotinic acid | 10.0 |
| Thiamine | 2.5 |
| Others | |
| Sucrose | 12500.0 |
| Dipotassium | 1500.0 |
| hydrogen | |
| phosphate | |
| Magnesium | 123.0 |
| sulfate | |
| Cupric chloride | 0.2 |
| Ferric chloride | 11.0 |
| Manganese | 0.4 |
| chloride | |
| Zinc sulfate | 0.8 |
| (anhydrous) | |

Adjusted to pH 6.8

Controlling value (%)={1−(Cb×Tai)/(Cai×Tb)}×100

Letters in the equation represent the following meanings.

Cb: Number of surviving worms before treatment in non-treating section

Cai: Number of surviving worms at observation in non-treated section

Tb: Number of surviving worms before treatment in non-treated section

Tai: Number of surviving worms at observation in a treated section

It may be said that a test agent exhibiting a significantly high controlling value has the pesticidal activity. More preferably, it may be determined that a test agent having the controlling value of 30% or more has substantial pesticidal activity, and it may be determined that a test agent having the controlling value of less than 30% has no substantial pesticidal activity.

The pesticidal agent in the present invention contains a chemical substance having an ability to modulate the activity of insect peptidyl-dipeptidase A or an agriculturally acceptable salt thereof as an active ingredient.

In the present invention, an agriculturally acceptable salt refers to a salt in such a form that preparation of a controlling agent and application of the preparation do not become impossible, and may be a salt in any form. Specifically, examples of the salt include acid addition salts with mineral acids such as hydrochloric acid, hydrobromic acid, hydriodic acid, sulfuric acid, nitric acid, and phosphoric, organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethansulfonic acid, or acidic amino acids such as aspartic acid and glutamic acid; salts with inorganic bases such as sodium, potassium, magnesium, and aluminum, organic bases such as methylamine, ethylamine, and ethanolamine, or basic amino acids with lysine and ornithine; and an ammonium salts.

In the present invention, the “pesticidal agent which comprises a substance having an ability to modulate the activity of an insect peptidyl-dipeptidase A or a an agriculturally acceptable salt thereof as an active ingredient” means an agent which can control pests by containing a substance having an ability to modulate the activity of insect peptidyl-dipeptidase A identified in the measuring method or an agriculturally acceptable salt thereof as an active ingredient. Preferable examples of the substance include a compound having an ability to inhibit a reaction of a peptidyl-dipeptidase A with the o-aminobenzoylglycyl-p-nitro-L-phenylalanyl-L-proline. More preferable examples of the substance include a substance having an ability to inhibit the reaction of the insect peptidyl-dipeptidase A with o-aminobenzoylglycyl-p-nitro-L-phenylalanyl-L-proline in a cell-free system, wherein in the presence of the substance of 10 μM or more the activity of the peptidyl-dipeptidase A is lower than that in the absence of the substance. In addition, further preferable examples of the substance include a substance having an ability to inhibit a reaction of the insect peptidyl-dipeptidase A with o-aminobenzoylglycyl-p-nitro-L-phenylalanyl-L-proline in a cell-free system with an IC50 of 100 μM or less.

In the present invention, the “method for assaying pesticidal activity of a test substance, which comprises a first step of measuring the activity of a peptidyl-dipeptidase A selected from the group A in a reaction system in which the peptidyl-dipeptidase A contacts with a test substance, and a second step of evaluating the pestcidal activity of the test substance based on the difference obtained by comparing the activity measured in the first step with the activity of a control” indicates a method characterized by comprising the first step and the second step in various methods for assaying a pesticidal ability of a test substance.

Herein, the group A indicates:

(a) a protein comprising the amino acid sequence of SEQ ID NO: 1;

(b) a protein comprising an amino acid sequence with deletion, addition or substitution of one or more amino acids in the amino acids sequence of SEQ ID NO:1, wherein the protein has peptidyl-dipeptidase A activity;

(c) a protein comprising an amino acid sequence that has sequence identity of 65% or more to the amino acid sequence of SEQ ID NO:1, wherein the protein has peptidyl-dipeptidase A activity;

(d) a protein comprising the amino acid sequence encoded by the nucleotide sequence of SEQ ID NO:2;

(e) a protein comprising an amino acid sequence encoded by a nucleotide sequence that has sequence identity of 65% or more to the nucleotide sequence of SEQ ID NO:2, wherein the protein has peptidyl-dipeptidase A activity;

(f) a protein comprising an amino acid sequence encoded by a polynucleotide, wherein the polynucleotide hybridizes under a stringent condition to a polynucleotide comprising a nucleotide sequence complementary to the nucleotide sequence of SEQ ID NO:2, and wherein the protein has peptidyl-dipeptidase A activity;

(g) a protein comprising an amino acid sequence of an insect peptidyl-dipeptidase A;

(h) a protein comprising an amino acid sequence of (f) aphid peptidyl-dipeptidase A (hereinafter, referred to as group A).

The first step is a step of measuring the activity of a peptidyl-dipeptidase A in the state where a peptidyl-dipeptidase A is contacted with a test substance by adding the test substance to the aforementioned various peptidyl-dipeptidase A activity measuring reaction systems. In addition, the second step is a step of comparing the activity at measurement of a test substance with the substance of a control, and evaluating a pesticidal ability based on the difference. Herein, a control means, for example, in the case where a test substance dissolved in a solvent is added to the reaction system, a test section in which only a solvent same as that used to dissolve the test substance is added.

A peptidyl-dipeptidase A used in a method for assaying a pestcidal ability possessed by a test substance, having the first step and the second step, is a protein shown in the group A. Among proteins of the group A, a difference which can be recognized between an amino acid sequence of protein represented by (a) and amino acid sequences of proteins represented by (b), (c), (e), (f), (g) and (h) is deletion, substitution, addition or the like of a part of amino acids. These include, for example, deletion due to processing which the protein having an amino acid sequence represented by (a) undergoes in a cell. In addition, examples include deletion, substitution, addition and the like of an amino acid generated by naturally occurring gene mutation due to a spices difference or an individual difference of an organism from which the protein is derived, or gene mutation which is artificially introduced by a site-directed mutagenesis, a random mutagenesis, mutation treatment or the like.

The number of amino acids undergoing the deletion, substitution, addition or the like may be the number in a range that the peptidase activity of a peptidyl-dipeptidase A can be found out. In addition, examples of substitution of an amino acid include substitution with an amino acid which is similar in characteristic in hydrophobicity, charge, pH and steric structure. Specific examples of the substitution include substitution in an group of (1) glycine, alanine; (2) valine, isoleucine, leucine; (3) aspartic acid, glutamic acid, asparagine, glutamine, (4) serine, threonine; (5) lysine, arginine; (6) phenylalanine, tyrosine and the like.

Examples of a procedure of artificially introducing the deletion, addition or substitution of an amino acid (hereinafter, collectively referred to as alteration of amino acid in some cases) include a procedure of introducing site-directed mutation into a DNA encoding an amino acid sequence represented by (a) and, thereafter, expressing this DNA by a conventional method. Herein, examples of a site-directed mutagenesis include a method utilizing amber mutation (gapped duplex method, Nucleic Acids Res., 12, 9441-9456 (1984)), a method by PCR using primers for mutation introduction, and the like. In addition, examples of a procedure of artificially altering an amino acid include a procedure of randomly introducing mutation into a DNA encoding an amino acid sequence represented by (a) and, thereafter, expressing this DNA by a conventional method. Herein, examples of a method of randomly introducing mutation include a method of performing PCR using a DNA encoding any of the aforementioned amino acid sequences as a template, and using a primer pair which can amplify each full length DNA at reaction condition under which an addition amount of each of dATP, dTTP, dGTP and dCTP used as a substrate is changed from a conventional concentration, or at reaction condition under which a concentration of Mg²⁺ promoting a polymerase reaction is increased from a conventional concentration. Examples of the procedure of PCR include a method described, for example, in Method in Molecular Biology, (31), 1994, 97-112. Another example includes a method described in WO 0009682.

Herein, the “sequence identity” refers to identity between two nucleotide sequences or two amino acids. The “sequence identity” is determined by comparing two sequences which are aligned in the optimal state over an all region of sequences to be compared. Herein, in optimal alignment of nucleotide sequences or amino acid sequences to be compared, addition or deletion (e.g. gap and the like) may be permitted. The sequence identity can be calculated by performing homology analysis to produce alignment using a program such as FASTA[Pearson & Lipman, Proc. Natl. Acad. Sci. USA, 4, 2444-2448 (1988)], BLAST [Altschul et al., Journal of Molecular Biology, 215, 403-410 (1990)], CLUSTAL W[Thompson, Higgins&Gibson, Nucleic Acid Research, 22, 4673-4680(1994a)] and the like. The program is generally available at the website (http://www.ddbj.nig.ac.jp) of DNA Data Bank of Japan (International DNA Data Bank managed in National Institute of Genetics, Center for Information Biology and DNA Data Bank of Japan; CIB/DDBJ). Alternatively, sequence identity can be also obtained using a commercially available sequence analyzing software. Specifically, for example, sequence identity can be calculated by performing homology analysis using GENETYX-WIN Ver. 5-(manufactured by Software Development Co. Ltd.) by a Lipman-Pearson method [Lipman, D. J. and Pearson, W. R., Science, 227, 1435-1441, (1985)] and producing alignment.

Examples of the “stringent condition” described in (f) include condition under which, in hybridization performed according to a conventional method described in Sambrook J., Frisch E. F., Maniatis T., Molecular Cloning 2nd edition, Cold Spring Harbor Laboratory press, for example, a hybrid is formed at 45° C. in a solution containing 6×SSC (a solution containing 1.5 m NaCl and 0.15 m trisodium citrate is 10×SSC) and, thereafter, this is washed with 2×SSC at 50° C. (Molecular Biology, John Wiley & Sons, N.Y. (1989), 6.3.1-6.3.6). A salt concentration in a washing step can be selected from condition from 2×SSC (low stringent condition) to 0.2×SSC (high stringent condition). A temperature in a washing step can be selected, for example, from condition from room temperature (low stringent condition) to 65° C. (high stringent condition). Alternatively, both of a salt concentration and a temperature can be changed.

A protein described in (h) indicates a peptidyl-dipeptidase A presents in a cotton aphid among an insect peptidyl-dipeptidase A, and includes a protein comprising an amino acid sequence described in (a).

In addition, a protein of the group A includes a protein comprising an amino acid sequence of an insect peptidyl-dipeptidase A described in (g) and, more preferably includes, a protein in which when aligned with the amino acid sequence of SEQ ID NO: 1 so that maximum sequence identity is obtained, amino acid residues at positions corresponding to (I) position 478, (II) position 498, (III) position 507 and (IV) position 567 of the amino acid sequence of SEQ ID NO: 1 are (I) cysteine (at position corresponding to 478), (II) glutamic acid (at position corresponding to 498), (III) tyrosine (at position corresponding to 507), and (IV) methionine (at position corresponding to 567), respectively. Herein, the “aligned with the amino acid sequence of SEQ ID NO: 1 so that maximum sequence identity is obtained” means that sequence identity of a plurality of amino acid sequences to be analyzed including the amino acid sequence of SEQ ID NO: 1 is analyzed by a program such as FASTA, BLAST, CLUSTAL W described above, and they are aligned. By aligning a plurality of sequences by the method, positions of conserved amino acid residues in each amino acid sequence can be determined regardless of insertion or deletion in an amino acid sequence. It is thought that conserved amino acid residues are present at the same position in the three-dimensional structure of the proteins of interest, and it is presumed that similar effect is possessed regarding specific function of the proteins of interest. For example, when insect peptidyl-dipeptidase A including the peptidyl-dipeptidase A sequence of which is disclosed in the present invention, are aligned with the amino acid sequence of SEQ ID NO: 1 so that maximum sequence identity of amino acid sequences is obtained, it is shown that amino acid residues at positions corresponding to (I) position 478, (II) position 498, (III) position 507 and (IV) position 567 of the amino acid sequence of SEQ ID NO: 1 are (I) cysteine (at position corresponding to 478), (II) glutamic acid (at position corresponding to 498), (III) tyrosine (at position corresponding to 507), and (IV) methionine (at position corresponding to 567), respectively.

A substance having a pesticidal ability can be screened by using a method of assaying a pesticidal ability by measuring a pesticidal ability or controlling effect on the aforementioned pests.

Alternatively, a substance having a pesticidal ability can be also screened by the method of assaying a pesticidal ability using a peptidyl-dipeptidase A. Specifically, when it has been identified that a pesticidal ability of a test substance is a certain value or more, or a certain value or less using the method of assaying a pesticidal ability using a peptidyl-dipeptidase A, a substance having a pesticidal ability can be screened by selecting the substance.

Since a substance selected by the screening method has a pesticidal ability, it can be used as a pesticidal agent containing the substance or an agriculturally acceptable salt as an active ingredient.

Control of pests can be usually performed by application an effective amount of a pesticidal agent to a crop protected, a pest, or a habitat of a pest.

When a pesticidal agent is used for agriculture and forestry, its application amount is usually 0.1 to 1000 g in terms of an amount of a pesticidal agent per 1000 m². When a pesticidal agent is formulated into an emulsion, a water-dispersible powder, a flowable preparation, a microcapsule preparation or the like, the agent is usually applied by diluting with water to an active ingredient concentration of 1 to 10,000 ppm, and spraying this and, when a pesticidal agent is formulated into a granule, a powder or the like, the agent is usually applied as it is.

A pesticidal agent can be used by foliage-treating a plant such as a crop and the like which should be protected from pests, and can be also used by treating a seedbed before a plantlet of a crop is transplanted, or a planting hole or a strain base at planting. Further, for the purpose of controlling pests habiting a soil of a cultivating land, the agent may be used by treating the soil. Alternatively, the agent may be used by a method of winding a resin preparation which has been processed to a sheet or a string, on a crop, stretching the preparation near a crop and/or spreading on a soil surface of a strain base.

When a pesticidal agent is used as a pest controlling agent for preventing an epidemic, an emulsion, a water-dispersible powder, a flowable or the like is usually applied by diluting with water so that an active ingredient concentration becomes 0.01 to 10,000 ppm, and an oily agent, an aerosol, a fumigant, a poison bait or the like is applied as it is.

Examples of one utility of a pesticidal agent include control of an external parasite of a livestock such as cattle, sheep, goat, and chicken, or a small animal such as dog, cat, rat, and mouse, in this case, the agent can be administered to an animal by the veterinarily known method. As a specific administration method, when systemic control is intended, the agent is administered, for example, by a tablet, mixing in feed, suppository, injection (intramuscular, subcutaneous, intravenous, intraperitoneal etc.) and the like, when non-systemic control is intended, the agent is used by a method of spraying an oily agent or an aqueous liquid agent, performing pour on or spot on treatment, washing an animal with a shampoo preparation or attaching a resin preparation which has been processed into a necklace or a ear tag to an animal. An amount of a pesticidal agent when administered to an animal body is usually in a range of 0.1 to 1,000 mg as expressed by total amount of a compound A and a compound B per 1 kg of an animal.

An application amount and an application concentration of them are both different depending on the situations such as a kind of a preparation, an application time, an application place, an application method, a kind of a pest, a damage degree and the like, can be increased or decreased regardless of the aforementioned range, and can be appropriately selected.

The aforementioned pesticidal agent can be used in the method of controlling pests as described above.

In addition, on the other hand, a pest can be also controlled by identifying a substance having a pesticidal ability evaluated by the aforementioned method of assaying a pesticidal ability possessed by a pest substance, having a first step and a second step using a peptidyl-dipeptidase A selected from group A, and contacting the identified substance having a pesticidal ability with a pest. Herein, as a method of contacting an identified substance having a pesticidal ability with a pest, the aforementioned preparation method, application method and the like can be used.

An amino acid sequence shown in the group B is an amino acid sequence of insect peptidyl-dipeptidase A comprising any amino acid sequence of the following (a) to (g).

(a) the amino acid sequence of SEQ ID NO: 1;

(b) an amino acid sequence with deletion, addition or substitution of one or more amino acids in the amino acid sequence of SEQ ID NO: 1, wherein the amino acid sequence has peptidyl-dipeptidase A activity;

(c) an amino acid sequence that has sequence identity of 65% or more to the amino acid sequence of SEQ ID NO: 1, wherein the amino acid sequence has peptidyl-dipeptidase A activity;

(d) the amino acid sequence encoded by the nucleotide sequence of SEQ ID NO: 2;

(e) an amino acid sequence encoded by a nucleotide sequence that has sequence identity of 65% or more to the nucleotide sequence of SEQ ID NO: 2, wherein the amino acid sequence has peptidyl-dipeptidase A activity;

(f) an amino acid sequence encoded by a polynucleotide, wherein the polynucleotide hybridizes under a stringent condition to a polynucleotide comprising a nucleotide sequence complementary to the nucleotide sequence of SEQ ID NO: 2, wherein the amino acid sequence has peptidyl-dipeptidase A activity; and

(g) an amino acid sequence of (f) aphid peptidyl-dipeptidase A.

Among amino acid sequences of the group B, a difference which can be recognized between an amino acid sequence represented by (a) and amino acid sequences represented by (b), (c), (e), (f) and (g) is deletion, substitution, addition or the like of a part of amino acids. These include, for example, deletion due to processing which the protein having an amino acid sequence represented by (a) undergoes in a cell. In addition, examples include deletion, substitution, addition and the like of an amino acid generated by naturally occurring gene mutation due to a spices difference or an individual difference of an organism from which the protein is derived, or gene mutation which is artificially introduced by a site-directed mutagenesis, a random mutagenesis, mutation treatment or the like.

The number of amino acids undergoing the deletion, substitution, addition or the like may be the number in a range that the peptidase activity of a peptidyl-dipeptidase A can be found out. In addition, examples of substitution of an amino acid include substitution with an amino acid which is similar in characteristic in hydrophobicity, charge, pH and steric structure. Specific examples of the substitution include substitution in an group of (1) glycine, alanine; (2) valine, isoleucine, leucine; (3) aspartic acid, glutamic acid, asparagine, glutamine, (4) serine, threonine; (5) lysine, arginine; (6) phenylalanine, tyrosine and the like.

Examples of a procedure of artificially introducing the deletion, addition or substitution of an amino acid (hereinafter, collectively referred to as alteration of amino acid in some cases) include a procedure of introducing site-directed mutation into a DNA encoding an amino acid sequence represented by (a) and, thereafter, expressing this DNA by a conventional method. Herein, examples of a site-directed mutagenesis include a method utilizing amber mutation (gapped duplex method, Nucleic Acids Res., 12, 9441-9456 (1984)), a method by PCR using primers for mutation introduction, and the like. In addition, examples of a procedure of artificially altering an amino acid include a procedure of randomly introducing mutation into a DNA encoding an amino acid sequence represented by (a) and, thereafter, expressing this DNA by a conventional method. Herein, examples of a method of randomly introducing mutation include a method of performing PCR using a DNA encoding any of the aforementioned amino acid sequences as a template, and using a primer pair which can amplify each full length DNA at reaction condition under which an addition amount of each of dATP, dTTP, dGTP and dCTP used as a substrate is changed from a conventional concentration, or at reaction condition under which a concentration of Mg²⁺ promoting a polymerase reaction is increased from a conventional concentration. Examples of the procedure of PCR include a method described, for example, in Method in Molecular Biology, (31), 1994, 97-112. Another example includes a method described in WO 0009682.

Herein, the “sequence identity” refers to identity between two nucleotide sequences or two amino acids. The “sequence identity” is determined by comparing two sequences which are aligned in the optimal state over an all region of sequences to be compared. Herein, in optimal alignment of nucleotide sequences or amino acid sequences to be compared, addition or deletion (e.g. gap and the like) may be permitted. The sequence identity can be calculated by performing homology analysis to produce alignment using a program such as FASTA[Pearson & Lipman, Proc. Natl. Acad. Sci. USA, 4, 2444-2448 (1988)], BLAST [Altschul et al., Journal of Molecular Biology, 215, 403-410 (1990)], CLUSTAL W[Thompson, Higgins&Gibson, Nucleic Acid Research, 22, 4673-4680(1994a)] and the like. The program is generally available at the website (http://www.ddbj.nig.ac.jp) of DNA Data Bank of Japan (International DNA Data Bank managed in National Institute of Genetics, Center for Information Biology and DNA Data Bank of Japan; CIB/DDBJ). Alternatively, sequence identity can be also obtained using a commercially available sequence analyzing software. Specifically, for example, sequence identity can be calculated by performing homology analysis using GENETYX-WIN Ver. 5 (manufactured by Software Development Co. Ltd.) by a Lipman-Pearson method [Lipman, D. J. and Pearson, W. R., Science, 227, 1435-1441, (1985)] and producing alignment.

Examples of the “stringent condition” described in (f) include condition under which, in hybridization performed according to a conventional method described in Sambrook J., Frisch E. F., Maniatis T., Molecular Cloning 2nd edition, Cold Spring Harbor Laboratory press, for example, a hybrid is formed at 45° C. in a solution containing 6×SSC (a solution containing 1.5 m NaCl and 0.15 m trisodium citrate is 10×SSC) and, thereafter, this is washed with 2×SSC at 50° C. (Molecular Biology, John Wiley & Sons, N.Y. (1989), 6.3.1-6.3.6). A salt concentration in a washing step can be selected from condition from 2×SSC (low stringent condition) to 0.2×SSC (high stringent condition). A temperature in a washing step can be selected, for example, from condition from room temperature (low stringent condition) to 65° C. (high stringent condition). Alternatively, both of a salt concentration and a temperature can be changed.

A protein having an amino acid sequence described in (g) indicates a peptidyl-dipeptidase A presents in a cotton aphid among an insect peptidyl-dipeptidase A, and includes a protein comprising an amino acid sequence described in (a).

A protein having an amino acid sequence shown in the group B can be prepared, for example, according to a method described later using a polynucleotide encoding an amino acid sequence shown in the group B.

An insect peptidyl-dipeptidase A can be used as a reagent that provides an indicator to evaluate a pesticidal activity. Specifically, for example, an insect peptidyl-dipeptidase A can be used as a reagent that provides an indicator to evaluate a pesticidal activity by using as a peptidyl-dipeptidase A used in the method of assaying a pesticidal ability using a peptidyl-dipeptidase A. In addition, a more specific method can be performed according to the aforementioned method of measuring the activity of a peptidyl-dipeptidase A.

In addition, when an insect peptidyl-dipeptidase A is used as a reagent that provides an indicator to evaluate a pesticidal activity, more preferably, it is desirable that an insect peptidyl-dipeptidase A is a peptidyl-dipeptidase A having an amino acid sequence shown in the group B.

A polynucleotide having a nucleotide sequence encoding an amino acid sequence shown in the group B (hereinafter, referred to as polynucleotide group B in some cases) has a nucleotide sequence from which a protein having an amino acid sequence can be produced shown in the group B, in a cell of an organism or an in vitro translation system. A polynucleotide group B may be a DNA cloned from a nature, a DNA in which deletion, substitution or addition of a nucleotide is introduced into a DNA cloned from a nature, for example, by a site-directed mutagenesis or a random mutagenesis, or an artificially synthesized DNA. Specifically, examples include a polynucleotide comprising a nucleotide sequence represented by SEQ ID NO: 2.

### <First Obtaining Method>

For example, a method of obtaining a polynucleotide comprising the nucleotide sequence of SEQ ID NO: 2 included in the polynucleotide group B will be shown below. As a step, total RNA is obtained from cotton aphids, cDNA library is synthesized, and PCR amplification is performed, thereby, a polynucleotide of interest can be obtained.

A population of adults and larvae of Aphis gossypii, which have been reared on leaves of potted cucumber, is scraped from the surface of the leaves with a small brush, and 630 mg of the obtained population is crushed into a powder in liquid nitrogen using a mortar and a pestle. From the resulting frozen crushed powder, RNA is isolated using a RNA extracting reagent ISOGEN (manufactured by Nippon Gene) as follows. After 10 ml of ISOGEN is added to the frozen crushed powder in the mortar, the crushed powder is ground for 10 minutes while kept on ice. After grinding, a fluid sample is transferred to a 15 ml tube with a pipette, and 2 ml of chloroform (manufactured by Wako Pure Chemical Industries, Ltd.) is added thereto. Immediately, the mixture is vigorously shaken for 15 seconds and then left at rest at room temperature for 3 minutes. Then, the resulting mixture is centrifuged at 12,000×g at 4° C. for 15 minutes, and each 5 ml of aqueous layer are transferred to two new tubes. After 5 ml of ISOGEN is added to each tube, the mixture was immediately shaken vigorously for 15 seconds, and left at rest at room, temperature for 3 minutes. Then, the resulting mixture is centrifuged at 12,000×g at 4° C. for 15 minutes, and each 10 ml of aqueous layer are transferred to new 50 ml tubes, respectively. Subsequently, 10 ml of isopropanol (manufactured by Wako Pure Chemical Industries, Ltd.) is added to each tube, and the mixture is kept on ice for 30 minutes. The resulting mixture is centrifuged at 12,000×g at 4° C. for 10 minutes to precipitate RNA. After the supernatant is removed, 20 ml of 70% ethanol is added to the residue. The resulting mixture is centrifuged at 10,000×g at 4° C. for 5 minutes. After the supernatant is removed, the precipitate of total RNA is slightly dried and then dissolved in 1 ml of commercially available RNase-free water (Nacalai Tesque, Inc.). An absorbance of the prepared total RNA is measured at 260 nm to calculate a concentration according to a conventional method.

RT-PCR is performed employing total RNA of cotton aphid obtained by the aforementioned method as a template, and using random primers (manufactured by Invitrogen) and superscript III (manufactured by Invitrogen) according to the manual annexed to the reagent, to synthesized a cDNA library.

PCR is performed employing cDNA library of cotton aphid obtained by the aforementioned method as a template, and using an oligonucleotide primer comprising the nucleotide sequence of SEQ ID NO: 3 and an oligonucleotide primer comprising the nucleotide sequence of SEQ ID NO: 4 as well as a PfuUtra High Fidelity polymerase (manufactured by Stratagene) according to the manual annexed to the reagent. The conditions of the PCR are as follows: incubation at 94° C. for 10 minutes; followed by 40 cycles of PCR, one cycle being 94° C. for 15 seconds, 60° C. for 15 seconds and 72° C. for 3 minutes; and followed by incubation at 72° C. for 7 minutes.

As described above, a polynucleotide comprising the nucleotide sequence of SEQ ID NO: 2 can be obtained.

### <Second Obtaining Method>

Alternatively, a polynucleotide shown in the polynucleotide group B can be also obtained by preparing a polynucleotide with mutation introduced therein by a method utilizing amber mutation which is the aforementioned site-directed mutagenesis, a method by PCR using a primer for introducing mutation or the like, using as a template a polynucleotide comprising the nucleotide sequence of SEQ ID NO: 2.

### <Third Obtaining Method>

Alternatively, a polynucleotide shown in the polynucleotide group B can be also obtained by a hybridization method using a polynucleotide comprising the nucleotide sequence of SEQ ID NO: 2 as a probe. More specifically, the third obtaining method can be performed according to a conventional hybridization described in the aforementioned Sambrook J., Frisch E. F., Maniatis T., Molecular Cloning 2nd edition, published by Cold Spring Harbor Laboratory press.

### <Fourth Obtaining Method>

Alternatively, a polynucleotide shown in the polynucleotide group B can be also obtained by preparing a primer based on an amino acid sequence of the known insect peptidyl-dipeptidase A and performing PCR. For isolation of homologues of peptidyl-dipeptidase A gene from other insect species such as German cockroach (Blatella germanica), degenerate primers are designed using Codehop program (publicly accessible on the website of Blocks Protein Analysis Server operated within the Fred Hutchinson Cancer Research Center at http://blocks.fhcrc.org/blocks/codehop.html), and based on the sequence of the aforementioned cotton aphid-derived peptidyl-dipeptidase A gene and the previously-known nucleotide sequences of silkworm gene (NCBI accession number AB026110.1), Anopheles gambiae gene (AAAB01008980), Drosophila melanogaster gene (NP_{—}477046.1, AAN10856, AAF52693) and Haematobia irritans gene (Q10715).

Partial sequences of a homologue of peptidyl-dipeptidase A gene in a selected insect species are amplified by a series of PCR using first-strand cDNA derived from the insect species as a template. Herein, the first-strand cDNA as a template is prepared by the aforementioned method using Superscript III. Amplification by PCR is performed using a set of degenerate primers as a forward primer and a reverse primer as well as Amplitaq Gold (manufactured by Applied Biosystems) according to the manufacturer's procedure annexed to the reagent. PCR conditions are 94° C. for 10 minutes; followed by 40 cycles of PCR, one cycle being 94° C. for 30 seconds, 45° C. for 1 minute, and 72° C. for 1 minute per 1 kb of a length of a predicted amplification product; and followed by 72° C. for 7 minutes. The PCR product is analyzed and purified by agarose gel electrophoresis to obtain DNA of interest. Further, the obtained DNA is cloned into the pCR-XL-TOPO vector (manufactured by Invitrogen), and sequenced.

Then, primers specific for the resulting partial sequences of the insect homologue of peptidyl-dipeptidase A gene are designed, and 3′RACE PCR or 5′RACE PCR is performed in order to obtain a full-length sequence of the gene. 3′ and 5′RACE PCRs are performed employing first-strand cDNA prepared from the insect total RNA as a template and using SMART PCR cDNA Synthesis Kit (manufactured by Clontech) according to the manufacturer's instructions annexed to the kit.

In 3′RACE and 5′RACE reactions, universal primer mix (UPM) contained in SMART PCR cDNA Synthesis Kit is used in combination with a forward primer or a reverse primer which is specific for the sequence of interest. PCR conditions are 1 cycle at 94° C. for 10 minutes; followed by 40 cycles of PCR, one cycle being 94° C. for 15 seconds, 63° C. for 15 seconds, and 72° C. for 1 minute per 1 kb of a length of a predicted amplification product; and followed by 1 cycle at 72° C. for 7 minutes. The resulting PCR product is analyzed and purified by agarose gel electrophoresis to obtain DNA of interest. Further, the obtained DNA is cloned into the pCR-XL-TOPO vector (manufactured by Invitrogen), and sequenced.

When a distinct amplification product is not obtained by the first-round PCR, nested PCR is performed using the first-round PCR product as a template. As primers, NUP primer contained in SMART PCR cDNA Synthesis Kit is used in combination with a specific forward primer or a specific reverse primer which is designed to bind internal sequence of the first-round PCR product of interest. PCR conditions are 1 cycle at 94° C. for 10 minutes; followed by 40 cycles of PCR, one cycle being 94° C. for 15 seconds, 63° C. for 15 seconds, and 72° C. for 2 minutes; and followed by 1 cycle at 72° C. for 7 minutes. The resulting PCR product is analyzed and purified by agarose gel electrophoresis to obtain DNA of interest. Further, the obtained DNA is cloned into the pCR-XL-TOPO vector (manufactured by Invitrogen), and sequenced.

The above sequencing results reveal 5′-terminal sequence and 3′-terminal sequence, each encoding N-terminal region and C-terminal region of the insect peptidyl-dipeptidase A, respectively.

Thus, a polynucleotide shown in the polynucleotide group B can be obtained by PCR by preparing a primer based on an amino acid sequence of the known insect peptidyl-dipeptidase A.

A polynucleotide having a nucleotide sequence complementary to a polynucleotide sequence of the polynucleotide group B can be used for obtaining a polynucleotide shown in the polynucleotide group B using a hybridization method.

The obtaining method in the present invention comprises a step of detecting a desired polynucleotide by hybridization, a step of identifying the detected desired polynucleotide, and a step of recovering the identified desired polynucleotide. Each step will be explained specifically below.

A step of detecting a desired polynucleotide by hybridization, and a step of identifying the detected desired polynucleotide can be performed by using, as a probe, a polynucleotide having a nucleotide sequence having complementarity to a nucleotide sequence of a polynucleotide group B, according to the method described, for example, in “Molecular Cloning: A Laboratory Manual 2nd edition” (1989), Cold Spring Harbor Laboratory Press, “Current Protocols In Molecular Biology” (1987), John Wiley & Sons, Inc. ISBN0-471-50338-X and the like.

Specifically, for example, a DNA having a nucleotide sequence having complementarity to a nucleotide sequence represented by SEQ ID NO: 2 is labeled with a radioisotope or a fluorescently labeled by the known method using Random Primed DNA Labelling Kit (manufactured by Boehringer), Random Primer DNA Labelling Kit Ver. 2 (manufactured by TAKARA SHUZO Co., Ltd.), ECL Direct Nucleic Acid Labelling and Ditection System (manufactured by Amersham Biosciences), or Megaprime DNA-labelling system (manufactured by Amersham Biosciences), and this can be used as probe.

Examples of condition for hybridization include stringent condition, and specifically, examples include condition under which incubation is performed at 65° C. in the presence of 6×SSC (0.9M NaCl, 0.09M sodium citrate), a 5×Denhart's solution (0.1% (w/v) Ficoll 400, 0.1% (w/v) polyvinylpyrrolidone, 0.1% BSA), 0.5% (w/v) SDS and 100 μg/ml denatured salmon spermatozoon DNA, or in a DIG EASY Hby solution (Boehringer Mammheim) containing 100 μg/ml denatured salmon spermatozoon DNA, then, incubation is performed two times at room temperature for 15 minutes in the presence of 1×SSC (0.15 mNaCl, 0.015 m sodium citrate) and 0.5% SDFS and, further, incubation is performed at 68° C. for 30 minutes in the presence of 0.1×SSC (0.015 m NaCl, 0.0015 m sodium citrate) and 0.5% SDS. More specifically, for example, a probe labeled with ³²P can be made by employing a polynucleotide having a nucleotide sequence having complementarity to a nucleotide sequence of a polynucleotide group B as a template, using Megaprime DNA-labelling system (manufactured by Amersham Pharmacia Biotech) and using a reaction solution designated in a kit. Colony hybridization is performed using this probe according to a conventional method, incubation is performed at 65° C. in the presence of 6×SSC (0.9M NaCl, 0.09M sodium citrate), a 5×Denhart's solution (0.1% (w/v) Ficoll 400, 0.1% (w/v) polyvinylpyrrolidone, 0.1% BSA), 0.5% (w/v) SDS and 100 μg/ml denatured salmon spermatozoon DNA, or in a DIG EASY Hyb solution (Boehringer Mannheim) containing 100 μg/ml denatured salmon spermatozoon DNA, then, incubation is performed two times at room temperature for 15 minutes in the presence of 1×SSC (0.15 m NaCl, 0.015 m sodium citrate) and 0.5% SDS and, further, incubation is performed at 68° C. for 30 minutes in the presence of 0.1×SSC (0.015 m NaCl, 0.0015 m sodium citrate) and 0.5% SDS, thereby, (a colony containing) a hybridizing polynucleotide can be detected. Thus, a desired polynucleotide can be detected by hybridization, and the detected desired polynucleotide can be identified.

For recovering the identified desired polynucleotide, a plasmid DNA can be recovered from a colony containing the polynucleotide detected and identified by the aforementioned method, for example, according to a method such as the alkali method described in “Molecular Cloning: A Laboratory Manual 2nd edition” (1989), Cold Spring Harbor Laboratory Press. A nucleotide sequence of the recovered desired polynucleotide (plasmid DNA) can be confirmed by a Maxam Gilbert method (described, for example, in Maxam, A. M & W. Gilbert, Proc. Natl. Acad. Sci. USA, 74, 560, 1977 etc.) or a Sanger method (described, for example, in Sanger, F. & A. R. Coulson, J. Mol. Biol., 94, 441, 1975, Sanger, F, & Nicklen and A. R. Coulson., Proc. Natl. Acad. Sci. USA, 74, 5463, 1977 etc.). Thereupon, for example, commercially available Termo Seqenase II dye terminator cycle sequencing kit (manufactured by Amersham biosciences), Dye Terminator Cycle Sequencing FS Ready Reaction Kit (manufactured by Applied Biosystems) and the like can be used.

A polynucleotide having a partial nucleotide sequence of a nucleotide sequence of the polynucleotide group B or a nucleotide sequence complementary to the partial nucleotide sequence can be used for obtaining a polynucleotide shown in the polynucleotide group B using PCR. More specifically, examples include a polynucleotide comprising a nucleotide sequence represented by SEQ ID NO: 3 or 4. The obtaining method in the present invention includes a step of amplifying a desired polynucleotide by PCR, a step of identifying the amplified desired polynucleotide, and a step of recovering the identified desired polynucleotide. Each step will be specifically explained below.

In a step of amplifying a desired polynucleotide by PCR, specifically, a DNA designed and synthesized from a partial nucleotide sequence of a nucleotide sequence of a polynucleotide group B or a nucleotide sequence having complementarity to the partial nucleotide sequence, based on an about 20 bp to about 40 bp nucleotide sequence, for example, a nucleotide sequence selected from a nucleotide sequence represented by SEQ ID NO: 2 and a sequence having complementarity to a nucleotide sequence represented by SEQ ID NO: 2 can be used as a primer set. Examples of a primer set include a set of a polynucleotide comprising a nucleotide sequence represented by SEQ ID NO: 3 and a polynucleotide comprising a nucleotide sequence represented by SEQ ID NO: 4. A PCR reaction solution is prepared, for example, by adding a reaction solution designated by a commercially available PCR kit to a cDNA library prepared by the aforementioned method. Reaction condition can be changed depending on a primer set to be used, and for example, condition under which after incubation at 94° C. for 10 seconds, around 40 cycles is repeated, 1 cycle being 94° C. for 15 seconds, 60° C. for 15 seconds, and 72° C. for 3 minutes and, further, incubation is performed at 72° C. for 3 minutes, condition under which incubation is performed at 94° C. for 2 minutes, thereafter, incubation is performed at about 8° C. for 3 minutes and, thereafter, around 40 cycles is repeated, 1 cycle being 94° C. for 30 seconds, 55° C. for 30 seconds, and 72° C. for 4 minutes, or condition under which 5 to 10 cycles is performed, 1 cycle being incubation at 94° C. for 5 seconds and, then, 72° C. for 4 minutes and, further, around 20 to 40 cycles is performed, 1 cycle being incubation at 94° C. for 5 seconds and, then, 70° C. for 4 minutes, can be used. In the PCR, for example, PfuUltra High Fidelity polymerase (manufactured by Stratagene), Amplitaq Gold (manufactured by Applied Biosystems), Takara Heraculase (Trademark) (manufactured by TAKARA SHUZO Co., Ltd.), a DNA polymerase contained in Advantage cDNA PCR Kit (manufactured by Clonetech), TaKaRa Ex Taq (manufactured by TAKARA SHUZO Co., Ltd.), PLATINUM™ PCR SUPER Mix (manufactured by Lifetech Oriental) can be used.

Identification of a desired polynucleotide amplified by PCR can be performed by measuring a molecular weight by agarose gel electrophoresis according to the method described in “Molecular Cloning: A Laboratory Manual 2nd edition” (1989), Cold Spring Harbor Laboratory Press. In addition, regarding the amplified desired polynucleotide, a sequencing reaction is performed using a commercially available DNA sequencing reaction kit, for example, Dye Terminator Cycle Sequencing FS Ready Reaction Kit (manufactured by Applied Biosystems) according to a manual annexed to the kit, and the nucleotide is analyzed using a DNA sequencer 3100 (manufactured by Applied Biosystems), thereby, a nucleotide sequence of the amplification fragment can be read.

Examples of a method of recovering the identified desired polynucleotide include a method of purifying and recovering the aforementioned polynucleotide identified by agarose gel electrophoresis from an agarose gel according to the method described in “Molecular Cloning: A Laboratory Manual 2nd edition” (1989), Cold Spring Harbor Laboratory Press. In addition, the thus recovered polynucleotide or a desired polynucleotide amplified by PCR can be cloned into a vector according to a conventional method described in “Molecular Cloning: A Laboratory Manual 2nd edition” (1989), Cold Spring Harbor Laboratory Press, and “Current Protocols In Molecular Biology” (1987), John Wiley & Sons, Inc. ISBN0-471-50338-X. Examples of a vector to be used include pUCA119 (manufactured by TAKARA SHUZO Co., Ltd.), pTVA118N (manufactured by TAKARA SHUZO Co., Ltd.), pBluescriptII (manufactured by Toyobo Co., Ltd.), pCR2.1-TOPO (manufactured by Invitrogen) and the like. In addition, a nucleotide sequence of the cloned polynucleotide can be confirmed by a Maxam Gilbert method (described, for example, in Maxam, A. M & W. Gilbert, Proc. Natl. Acad. Sci. USA, 74, 560, 1977) or a Sanger method (described, for example, in Sanger, F. & A. R. Coulson, J. Mol. Biol., 94, 441, 1975, Sanger, F, & Nicklen and A. R. Coulson, Proc. Natl. Acad. Sci. USA, 74, 5463, 1977). Thereupon, for example, a commercially available Termo Seqenase II dye terminator cycle sequencing kit (manufactured by Amersham biosciences), Dye Terminator Cycle Sequencing FS Ready Reaction Kit (manufactured by Applied Biosystems) and the like can be used.

In addition, a polynucleotide having a partial nucleotide sequence of a nucleotide sequence of the polynucleotide group B or a nucleotide sequence complementary to the partial nucleotide sequence can be used for obtaining a polynucleotide shown in the polynucleotide group B using not only a PCR method, but also the aforementioned hybridization method. More specifically, examples include a polynucleotide comprising a nucleotide sequence represented by SEQ ID NO: 3 or 4.

Examples of a method for preparing a protein having an amino acid sequence shown in the group B include a method of culturing a transformant with a polynucleotide selected from a polynucleotide group B introduced therein, and recovering the produced protein. In addition, for preparing a transformant used herein, it is a work such as preparation of a circular polynucleotide containing a polynucleotide in which a polynucleotide selected from a polynucleotide group B is operably ligated to a baculovirus-derived promoter. The method will be explained in detail below.

In addition, a peptidyl-dipeptidase A shown in a group A which is used in the method of assaying a pesticidal ability using a peptidyl-dipeptidase A can be prepared and obtained by the similar method, using a polynucleotide having a nucleotide sequence encoding a peptidyl-dipeptidase A used.

Baculovirus is a virus belonging to a diverse group of large double-stranded DNA viruses that infect many different species of insects as their natural hosts. The baculovirus genome is replicated and transcribed in the nuclei of infected host cells where the large circular baculovirus DNA (between 80 and 200 kb) is packaged into rod-shaped nucleocapsids. Examples of isolates used in foreign gene expression are Autographa californica multiple nuclear polyhedrosis virus (AcMNPV) and Bombyx mori (silkworm) nuclear polyhedrosis virus (BmNPV).

A baculovirus-derived promoter means a promoter of a gene contained in a baculovirus genome. Among them, examples of a promoter of baculovirus used for expressing a foreign gene include a promoter of a polyhedrin gene, and a promoter of a p10 gene (Harris and Polayes (1997). Focus 19, 6-8).

A promoter of a polyhedrin (polyhedron) gene is a promoter of a gene encoding polyhedrin which is a main component of an intranuclear inclusion body produced when baculovirus infects an insect cell. Polyhedrin is not a protein necessary for replicating a virus and, by substituting its gene with a gene of a protein of interest, the protein of interest amounting to 50% of a cell protein may be expressed.

In the present invention, “operably linked” means that a polynucleotide containing a gene of interest is linked downstream of a polynucleotide containing a promoter sequence so that the gene of interest can be transcribed in a used transcription system. Specifically, for example, when a promoter of a polyhedrin gene described later is used, a polynucleotide containing a gene of interest may be linked downstream of a promoter of a polyhedrin gene. In addition, for example, when a promoter other than a polyhedrin gene promoter is used, it is also possible to link a polynucleotide containing a gene of interest downstream of a polynucleotide containing a promoter sequence other than a polyhedrin gene promoter. More specifically, for example, when a plasmid pFastbacHT (manufactured by Invitrogen) vector utilizing a polyhedrin gene promoter is used, the polynucleotide can be operably linked by ligating a gene of interest into a restriction enzyme site such as BamHI, EcoRI, SalI, SpeI, NotI, XbaI, PstI, XhoI, SphI, KpnI, and HindIII located downstream of a polyhedrin gene promoter.

In the present invention, the “circular polynucleotide” is a polynucleotide which has been made to be circular by binding of ends of the polynucleotide strand, and examples include chromosomal DNAs of many bacteria in addition to a plasmid DNA, a bacmid DNA and the like.

A plasmid DNA is a relatively low-molecular circular polynucleotide, and examples include pET (manufactured by Takara Mirus Bio Inc.) and pBluescriptII (manufactured by Stratagene), used for cloning and expression in E. coli. Additional examples include pFastBac1, pFastBac HT A, pFastBac HT B, pFastBac HT C, pFastBac Dual, pBlueBacII (manufactured by Invitrogen), pAcSG2 (manufactured by Pharmingen) and the like, which contain a baculovirus expression cassette.

The bacmid is a high molecular weight DNA that consists of a BAC (bacterial artificial chromosome) that contains the entire baculoviral genome, for example bMON14272 (136 kb) that is present in DH10Bac™ E. coli cells (invitrogen). Bacmid DNA propagates as a large plasmid in E. coli cells and may contain an expression cassette for expression of a foreign gene under control of a baculoviral promoter.

A circular polynucleotide in which a polynucleotide comprising a nucleotide sequence encoding an amino acid sequence shown in the group B is operably linked to a baculovirus-derived promoter is specifically, for example, a circular polynucleotide containing a DNA comprising a cotton aphid peptidyl-dipeptidase A gene operably linked to a polyhedrin promoter of baculovirus, and can be prepared and obtained, for example, according to the following method.

A plasmid DNA containing a cotton aphid peptidyl-dipeptidase A gene cloned according to the aforementioned method is digested with EcoRI and Xho1, and the resulting about 1.9 kbp DNA fragment comprising a cotton aphid peptidyl-dipeptidase A gene is ligated with a plasmid vector pFastBac HT B (manufactured by Invitrogen) which has been digested with EcoRI and Xho1 in advance. The obtained plasmid is one example of a circular polynucleotide containing a DNA comprising a cotton aphid peptidyl-dipeptidase A gene operably linked to a polyhedrin promoter of baculovirus. In addition, according to the manual annexed to Bac-to-Bac Baculovirus Expression System (manufactured by Invitrogen), this plasmid may be introduced into Escherichia coli DH10Bac, and a DNA containing a polyhedrin gene promoter and a cotton aphid peptidyl-dipeptidase A gene can be inserted into a bacmid DNA by a recombination in the cell. For example, by the aforementioned method, a circular polynucleotide containing a DNA comprising a cotton aphid peptidyl-dipeptidase A gene operably linked to a polyhedrin promoter of baculovirus can be prepared and obtained.

Similarly, a circular polynucleotide can be prepared by ligating a nucleotide encoding an amino acid sequence shown in the group B to a vector.

In the present invention, the “origin of replication” is the specific DNA sequence necessary for replicating itself in a host cell. Examples of origin of replication include colE1 and f1 for bacterial plasmids. In addition, a homologous repeated (hrs) region, and a non-hr region are present in a bacmid DNA (Pijlman et al. (2003) Journal of General Virology 84, 2669-2678).

One example of the circular polynucleotide is a baculovirus shuttle vector. Herein, a baculovirus shuttle vector means the bacmid DNA. The bacmid DNA can be propagated and genetically engineered in E. coli. Upon isolation from E. coli and introduction into an insect host cell, bacmids can be propagated as a virus. For example in the case of bMON14272 (invitrogen), the recombinant bacmids are generated in E. coli by transposition of a mini-Tn7 element, containing the baculoviral expression cassette from a pFastBac™ donor plasmid to the mini-attTn7 attachment site on the bacmid.

Insect cells to be used as host for the propagation of the baculovirus and for expression of the foreign protein by means of the recombinant baculovirus include cell lines derived from Spodoptera frugiperda or from Trichoplusia ni. Examples of such cell lines are, Sf21 cells, Sf9 cells, Tn-368 or High Five cells or Mimic Sf9 Insect cells (Invitrogen).

A transformant is a eukaryotic cell or a prokaryotic cell which has been genetically altered by introduction of a foreign polynucleotide into a cell. Examples of a transformant include an Escherichia coli cell transformed by introduction of a plasmid containing a baculovirus expression cassette such as plasmid vector pFastBac (manufactured by Invitrogen) and the like. In addition, examples of the technique of introducing a DNA into a host cell include transformation, transfection, protoplast fusion, lipofection, electroporation and the like.

Examples of a transformant in which a polynucleotide encoding an amino acid sequence shown in the group B is introduced include transformed Escherichia coli in which a DNA comprising a cotton aphid peptidyl-dipeptidase A gene operably linked to a polyhedrin promoter of baculovirus is introduced. Specifically, the transformant can be prepared according to the following method.

A transformant can be prepared by introducing a plasmid vector pFastBac HT B (manufactured by Invitrogen) in which a DNA containing a cotton aphid peptidyl-dipeptidase A gene is inserted between an EcoRI site and a XhoI site into an Escherichia coli cell according to the method described in “Molecular Cloning: A Laboratory Manual 2nd edition” (1989), Cold Spring Harbor Laboratory Press. In addition, a transformant can be also prepared by introducing the same plasmid vector into Escherichia coli DH10Bac according to a method described in a manual annexed to Bac-to-Bac Baculovirus Expression System (manufactured by Invitrogen).

Alternatively, a transformant can be also obtained by transfecting the bacmid DNA in which a fragment containing a polyhedrin gene promoter and a cotton aphid peptidyl-dipeptidase A gene is inserted, into an insect cell according to a method described in a manual annexed to Bac-to-Bac Baculovirus Expression System (manufactured by Invitrogen).

A recombinant baculovirus is baculovirus in which the sequence of the baculovirus genome has been altered by genetic-engineering technique.

Specific examples of recombinant baculovirus include recombinant baculovirus containing a DNA fragment comprising a cotton aphid peptidyl-dipeptidase A gene operably linked to a polyhedrin promoter of baculovirus. A recombinant baculovirus can be prepared, for example, by homologous recombination between baculoviral DNA and transfer vector DNA in insect cells (Kitts (1996) Cytotechnology 20, 111-123). Alternatively, recombinant baculovirus can be also prepared, for example, by introducing a recombinant bacmid DNA containing a DNA fragment comprising a cotton aphid peptidyl-dipeptidase A gene operably linked to a polyhedrin promoter of baculovirus prepared by the aforementioned method into insect cells. Specifically, recombinant baculovirus can be prepared by transfecting a recombinant bacmid DNA containing a DNA fragment comprising a cotton aphid peptidyl-dipeptidase A gene operably linked to the aforementioned polyhedrin promoter of baculovirus into an insect cell according to the method described in a manual annexed to Bac-to-Bac Baculovirus Expression System (manufactured by Invitrogen). More specifically, the recombinant bacmid DNA is transfected into Sf9 cells (ATCC: CRL-1711) according to the manual annexed to Bac-to-Bac Baculovirus (Trademark) Expression System (manufactured by Invitrogen) to obtain recombinant baculovirus. For the transfection, cellfectin (manufactured by Invitrogen), Grace's Insect Cell Culture Medium supplemented with L-amino acids (manufactured by Invitrogen, Gibco), 10% Foetal Bovine Serum (manufactured by Clontech), and penicillin/streptomycin (manufactured by Life Technologies) are used. In addition; for transfection, 2 μg of bacmid DNA and 7 μl of cellfectin are used. A P1 recombinant baculovirus stock is recovered after 8 days according to the manual annexed to Bac-to-Bac Baculovirus (Trademark) Expression System (manufactured by Invitrogen). For example, 0.5 ml of this baculovirus stock can be further propagated by inoculating on 300 ml of Sf9 cell cultures of 1×10⁶ cells/ml. The amplified baculovirus stock is harvested after 4 days according to the manual annexed to Bac-to-Bac Baculovirus (Trademark) Expression System (manufactured by Invitrogen). The Sf9 cell are suspension-cultured in Erlenmeyer flasks at 27° C. and at 135 rpm. A component of a medium used in this culturing include Grace's Insect Cell Culture Medium supplemented with L-amino acids (manufactured by Invitrogen, Gibco), 10% Foetal Bovine Serum (manufactured by Clontech), penicillin/streptomycin (manufactured by Life Technologies), and final consideration 0.1% Pluronic F-68 (manufactured by Sigma-aldrich).

A peptidyl-dipeptidase A can be prepared by culturing a transformant prepared by the aforementioned method, and recovering the produced insect-derived peptidyl-dipeptidase A.

Peptidyl-dipeptidase A protein may be produced by for example a recombinant baculovirus/Sf9 cell expression system. This system is one of the most powerful and versatile eukaryotic expression systems available, and may be used to express heterologous genes from many different sources, including fungi, plants, bacteria and viruses.

Alternatively, Peptidyl-dipeptidase A protein may be produced by a recombinant E. coli expression system. This system is the most frequently used prokaryotic expression system for the high-level production of heterologous proteins. E. coli is genetically and physiological the best characterized organism known, it is easy to manipulate, many tools are available, it is able to grow very fast, it grows on cheap complex or well-defined minimal media and it has an extremely high capacity to synthetize heterologous protein.

In addition, an insect-derived peptidyl-dipeptidase A produced by culturing a transformant is lysed by a method such as sonication, French press, and Dyno mill, and recovered in a form contained in a cell crude extract, and a purified protein can be obtained by using a procedure conventionally used in enzyme purification such as ion exchange column chromatography, reverse phase column chromatography, gel filtration column chromatography and the like. Alternatively, when it is devised that an insect-derived peptidyl-dipeptidase A is produced in a form with His-tag, a purified protein can be obtained rapidly from a cell crude extract by affinity column chromatography which specifically recognizes and binds to the His-tag. By the method, an insect-derived peptidyl-dipeptidase A can be prepared.

For example, an insect-derived peptidyl-dipeptidase A can be prepared by culturing a transformed insect cell with a DNA fragment containing a cotton aphid peptidyl-dipeptidase A gene operably linked to a polyhedrin promoter of baculovirus, and grinding the cell with a French press, followed by purification with column chromatography.

More specifically, for example, 4×10⁸ Sf9 cells are suspended in 30 ml of a stock solution of recombinant baculovirus containing a recombinant bacmid DNA containing a DNA fragment comprising a cotton aphid peptidyl-dipeptidase A gene operably ligated to a polyhedrin promoter of baculovirus, the suspension is rotation-cultured in 125 ml Erlenmeyer at 135 rpm, at 27° C. for 1 hour. Thereafter, each ⅓ of the cell suspension is placed into three 25 ml Erlenmeyer flasks, a medium is added to each flask so that a volume of a culturing solution becomes 100 ml, 1 ml of a 10% Pluronic solution is added, and this is cultured again. After 48 hours, Sf9 cells infected with baculovirus are harvested by centrifugation at 290×g for 5 minutes. Buffer A (50 mM Hepes-HaOH pH7.5, 0.5 m NaCl, 10 mM imidazole) is added to the harvested Sf9 cells to suspend them, and cells are lysed at a pressure of 1,500 psi using a French press (manufactured by Thermo Spectronic) according to a method described in the annexed manual. This French-pressed solution is centrifuged at 13,000×g at 4° C. for 20 minutes, and the resulting supernatant is filtered through a 0.45 mm filter. Then, this is injected into two HiTrap/HisTrap affinity columns (column volume 5 ml, manufactured by Amersham biosciences) connected in series which are equilibrated with buffer A (50 mM Hepes-HaOH pH7.5, 0.5 m NaCl, 10 mM imidazole), and columns are washed with 100 ml of buffer A. Then, columns are washed with 150 ml of a buffer obtained by mixing 93% of buffer A and 7% of buffer B (50 mM Hepes-HaOH pH7.5, 0.5 m NaCl, 500 mM imidazole). Finally, 60 ml of a buffer obtained by mixing 50% of buffer A and 50% of buffer B is injected into columns. Each 1 ml of the eluted fractions are fractionated, and stored, and an aliquot is analyzed with SDS-PAGE to identify fractions containing 75 Kda of a peptidyl-dipeptidase A. Those fractions are a solution containing a peptidyl-dipeptidase A at a large amount. Further, each 1.5 ml of solutions containing a peptidyl-dipeptidase A are injected into HiTrap desalting columns (column volume 5 ml, manufactured by Amersham biosciences) equilibrated with buffer C (50 mM Hepes-HaOH pH7.5, 0.5 m NaCl), then 4.5 ml of buffer C is injected, eluted fractions are recovered. This fraction contains an objective peptidyl-dipeptidase A dissolved in a buffer C. By analyzing an aliquot of this fraction with SDS-PAGE, it can be confirmed that 75 Kda of a peptidyl-dipeptidase A is contained.

An insect peptidyl-dipeptidase A comprising an amino acid sequence shown in the group B can be used as a research tool. For example, it can be used as a research tool for performing study such as assaying of the pestcidal ability, screening of a chemical substance having a pestcidal ability, and the like. In addition, for example, also in study of analyzing action and mechanism of an agent which acts on a peptidyl-dipeptidase A, a peptidyl-dipeptidase A can be utilized as a research tool.

In addition, polynucleotides encoding amino acid sequences shown in the group B and polynucleotides having a nucleotide sequence having complementarity to them, as well as partial nucleotide sequences of polynucleotides encoding amino acid sequences shown in the group B, or polynucleotides having nucleotide sequences having complementarity to the partial nucleotide sequences, and a polynucleotide complying a nucleotide sequence represented by SEQ ID NO: 3 or 4 can be used as a research tool. For example, a part of them functions as a polynucleotide used in a method of preparing a peptidyl-dipeptidase A as described above. In addition, a part can be used as an important research tool for performing obtaining a polynucleotide shown in a polynucleotide group B using PCR, or obtaining a polynucleotide shown in a polynucleotide group B using hybridization, as described above.

Particularly, upon implementation of screening of a pestcidal agent, they can be used as an experimental tool for an experiment which is performed for screening. Specifically, they can be used as an experimental tool for an experiment which is performed upon implementation of the assaying of a pestcidal ability, screening of a chemical substance having a pestcidal ability, and the like.

Further, the present invention also includes a system which comprises a means to input, store and manage data information of an ability of test substances, wherein said ability is an ability to modulate the activity of an insect peptidyl-dipeptidase A (hereinafter, referred to as means a in some cases), a means to query and retrieve the data information based on a desired criterion (hereinafter, referred to as means b in some cases), and a means to display and output the result which is queried and retrieved (hereinafter, referred to as means c in some cases) (hereinafter, referred to as present system in some cases).

First, a means a will be explained. A means a is a means to, after data information of an ability to modulate the activity of an insect-derived peptidyl-dipeptidase A possessed by the test substance is inputted, store and manage the inputted information, as described above. The information is inputted by an inputting means 1, and is usually memorized in a memory means 2. Examples of an inputting means include means which can input the information such as a keyboard and a mouse. When inputting and storing managing of the information are completed, a procedure progresses to a next means b. For storing managing the information, a large amount of data may be effectively stored and managed by inputting information having a data structure using a hardware such as a computer, and a software such as OS and database management, and storing the information into a suitable memory device, for example, computer-readable recording medium such as a flexible disc, a photomagnetic disc, CD-ROM, DVD-ROM, and a hard disc.

A means b will be explained. A means b is a means to query and retrieve the data information stored and managed by a means of a based on criterion for obtaining a desired result, as described above. For the information, when criterion for querying and retrieving is inputted by an inputting means 1, and information in conformity with the criterion is selected among the information usually memorized in a memory means 2, a procedure progresses to a next means c. The selected result is usually memorized in a memory means 2 and, further, can be displayed by a displaying outputting means 3.

A means c will be explained. A means c is a means to display and output the result which is queried and retrieved, as described above. Examples of the displaying outputting means 3 include a display, a printer and the like, and the result may be displayed on a display device of a computer, or may be outputted on a paper by printing.

### EXAMPLES

The present invention will be explained in more detail below by way of Examples, but the present invention is not limited to these particular Examples.

### Example 1

### Extraction of Total RNA from Cotton Aphid and German Cockroach

(1) Extraction of Total RNA from Cotton Aphid.

A population of adults and larvae of cotton aphid (Aphis gossypii), which had been reared on leaves of potted cucumber, was scraped from the surface of the leaves with a small brush, and 630 mg of the obtained population was crushed into a powder in liquid nitrogen using a mortar and a pestle. From the resulting frozen crushed powder, RNA was isolated using a RNA extracting reagent ISOGEN (manufactured by Nippon Gene) as follows. After 10 ml of ISOGEN was added to the frozen crushed powder in the mortar, the crushed powder was ground for 10 minutes while kept on ice. After grinding, a fluid sample was transferred to a 15 ml tube with a pipette, and 2 ml of chloroform (manufactured by Wako Pure Chemical Industries, Ltd.) was added thereto. Immediately, the mixture was vigorously shaken for 15 seconds and then left at rest at room temperature for 3 minutes. Then, the resulting mixture was centrifuged at 12,000×g at 4° C. for 15 minutes, and each 5 ml of aqueous layer were transferred to two new tubes. After 5 ml of ISOGEN was added to each tube, the mixture was immediately shaken vigorously for 15 seconds, and left at rest at room temperature for 3 minutes. Then, the resulting mixture was centrifuged at 12,000×g at 4° C. for 15 minutes, and each 10 ml of aqueous layer were transferred to new 50 ml tubes, respectively. Subsequently, 10 ml of isopropanol (manufactured by Wako Pure Chemical Industries, Ltd.) was added to each tube, and the mixture was kept on ice for 30 minutes. The resulting mixture was centrifuged at 12,000×g at 4° C. for 10 minutes to precipitate RNA. After the supernatant was removed, 20 ml of 70% ethanol was added to the residue. The resulting mixture was centrifuged at 10,000×g at 4° C. for 5 minutes. After the supernatant was removed, the precipitate of total RNA was slightly dried and then dissolved in 1 ml of commercially available RNase-free water (Nacalai Tesque, Inc.). A concentration of the prepared total RNA (calculated from an absorbance at 260 nm) was 6.9 mg/ml.

(2) Extraction of Total RNA from German Cockroach

Adults, nymphs and oothecae of artificially-reared German cockroach (Blattella germanica) were provided as samples. Ten (10) of adult males and 10 of adult females (individuals from each of which ootheca has been removed) were used as an adult sample of 1.1 g, 10 of nymph males and 10 of nymph females were used as a nymph sample of 1.0 g, and 26 oothecae were used as an ootheca sample of 1.0 g. Three kinds of these samples were separately crushed into a powder in liquid nitrogen using separate mortars and pestles. From each of the resulting frozen crushed powders, RNA was isolated using a RNA extracting reagent ISOGEN (manufactured by Nippon Gene) as follows. After 10 ml of ISOGEN was added to the frozen crushed powder in the mortar, the crushed powder was ground for 10 minutes while kept on ice. After grinding, a fluid sample was transferred to a 15 ml tube with a pipette, and 2 ml of chloroform (manufactured by Wako Pure Chemical Industries, Ltd.) was added thereto. Immediately, the mixture was vigorously shaken for 15 seconds and then left at rest at room temperature for 3 minutes. Then, the resulting mixture was centrifuged at 12,000×g at 4° C. for 15 minutes, and each 5 ml of aqueous layer were transferred to two new tubes. After 5 ml of ISOGEN was added to each tube, the mixture was immediately shaken vigorously for 15 seconds, and left at rest at room temperature for 3 minutes. Then, the resulting mixture was centrifuged at 12,000×g at 4° C. for 15 minutes, and each 10 ml of aqueous layer were transferred to new 50 ml tubes, respectively. Subsequently, 10 ml of isopropanol (manufactured by Wako Pure Chemical Industries, Ltd.) was added to each tube, and the mixture was kept on ice for 30 minutes. The resulting mixture was centrifuged at 12,000×g at 4° C. for 10 minutes to precipitate RNA. After the supernatant was removed, 20 ml of 70% ethanol was added to the residue. The resulting mixture was centrifuged at 10,000×g at 4° C. for 5 minutes. After the supernatant was removed, the precipitate of total RNA was slightly dried and then dissolved in 1 ml of commercially available RNase-free water (Nacalai Tesque, Inc.). A concentration of the prepared total RNA (calculated from absorbance at 260 nm) was 1.1 mg/ml in the case of adult-derived total RNA, was 2.5 mg/ml in the case of nymph-derived total RNA, and 1.4 mg/ml in the case of ootheca-derived total RNA.

### Example 2

### Isolation of Cotton Aphid Peptidyl-Dipeptidase A Gene

First-strand cDNA was prepared using total RNA from cotton aphid, random Primers (Invitrogen) and Superscript III (Invitrogen) for RT-PCR according to the manufacturer's procedure of Superscript III.

A full-length cDNA of cotton aphid peptidyl-dipeptidase A was amplified by PCR using an oligonucleotide comprising the nucleotide sequence of SEQ ID NO: 3 and an oligonucleotide comprising the nucleotide sequence of SEQ ID NO: 4, which are primers specific for the gene, and PfuUltra High Fidelity polymerase (manufactured by Stratagene) according to the manufacturer's procedure. First-strand cDNA, prepared as described above, was used as template. The PCR conditions used were as follows: an initial denaturation at 94° C. for 10 minutes; followed by 40 cycles of PCR, one cycle being 94° C. for 15 seconds, 60° C. for 15 seconds, and 72° C. for 3 minutes; followed by 72° C. for 7 minutes. The resulting PCR products were analyzed and purified by agarose gel electrophoresis to clone the 1921 bp DNA of interest, comprising the nucleotide of SEQ ID NO: 2, into the pCR-blunt vector (Invitrogen). The amino acid sequence presumed from the nucleotide sequence was the amino acid sequence of SEQ ID NO: 1. The resulting plasmid was named pGAT1.

### Example 3

### Isolation of German Cockroach Peptidyl-Dipeptidase Gene

For isolation of homologues of peptidyl-dipeptidase A gene from other insect species such as German cockroach (Blatella germanica), degenerate primers are designed using Codehop program (publicly accessible on the website of Blocks Protein Analysis Server operated within the Fred Hutchinson Cancer Research Center at http://blocks.fhcrc.org/blocks/codehop.html), and based on the sequence of the aforementioned cotton aphid-derived peptidyl-dipeptidase A gene and the previously-known nucleotide sequences of silkworm gene (NCBI accession number AB026110.1), Anopheles gambiae gene (AAAB01008980), Drosophila melanogaster gene (NP_{—}477046.1, AAN10856, AAF52693) and Haematobia irritans gene (Q10715).

Partial sequences of a homologue of peptidyl-dipeptidase A gene in a selected insect species are amplified by a series of PCR using first-strand cDNA derived from the insect species as a template. Herein, the first-strand cDNA as a template is prepared by the aforementioned method using Superscript III. Amplification by PCR is performed using a set of degenerate primers as a forward primer and a reverse primer as well as Amplitaq Gold (manufactured by Applied Biosystems) according to the manufacturer's procedure annexed to the reagent. PCR conditions are 94° C. for 10 minutes; followed by 40 cycles of PCR, one cycle being 94° C. for 30 seconds, 45° C. for 1 minute, and 72° C. for 1 minute per 1 kb of a length of a predicted amplification product; and followed by 72° C. for 7 minutes. The PCR product is analyzed and purified by agarose gel electrophoresis to obtain DNA of interest. Further, the obtained DNA is cloned into the pCR-XL-TOPO vector (manufactured by Invitrogen), and sequenced.

Thus, partial sequences of a peptidyl-dipeptidase A gene of Blatella germanica each comprising the nucleotide sequence of SEQ ID NO: 12, 14, 16, 18 or 20 were obtained. Amino acid sequence presumed from each of these partial sequences was the amino acid sequence of SEQ ID NO: 13, 15, 17, 19 or 21, respectively.

Then, primers specific for the resulting partial sequences of the insect homologue of peptidyl-dipeptidase A gene are designed, and 3′RACE PCR or 5′RACE PCR is performed in order to obtain a full-length sequence of the gene. 3 and 5′RACE PCRs are performed employing first-strand cDNA prepared from the insect total RNA as a template and using SMART PCR cDNA Synthesis Kit (manufactured by Clontech) according to the manufacturer's instructions annexed to the kit.

In 3′RACE and 5′RACE reactions, universal primer mix (UPM) contained in SMART PCR cDNA Synthesis Kit is used in combination with a forward primer or a reverse primer which is specific for the sequence of interest. PCR conditions are 1 cycle at 94° C. for 10 minutes; followed by 40 cycles of PCR, one cycle being 94° C. for 15 seconds, 63° C. for 15 seconds, and 72° C. for 1 minute per 1 kb of a length of a predicted amplification product; and followed by 1 cycle at 72° C. for 7 minutes. The resulting PCR product is analyzed and purified by agarose gel electrophoresis to obtain DNA of interest. Further, the obtained DNA is cloned into the pCR-XL-TOPO vector (manufactured by Invitrogen), and sequenced.

When a distinct amplification product is not obtained by the first-round PCR, nested. PCR is performed using the first-round PCR product as a template. As primers, NUP primer contained in SMART PCR cDNA Synthesis Kit is used in combination with a specific forward primer or a specific reverse primer which is designed to bind internal sequence of the first-round PCR product of interest. PCR conditions are 1 cycle at 94° C. for 10 minutes; followed by 40 cycles of PCR, one cycle being 94° C. for 15 seconds, 63° C. for 15 seconds, and 72° C. for 2 minutes; and followed by 1 cycle at 72° C. for 7 minutes. The resulting PCR product is analyzed and purified by agarose gel electrophoresis to obtain DNA of interest. Further, the obtained DNA is cloned into the pCR-XL-TOPO vector (manufactured by Invitrogen), and sequenced.

The above sequencing results reveal 5′-terminal sequence and 3′-terminal sequence, each encoding N-terminal region and C-terminal region of the insect peptidyl-dipeptidase A, respectively.

### Example 4

### Construction of Recombinant Bacmid

(1) Cloning of Peptidyl-Dipeptidase a cDNA into Gene Expression Vector pFastBac (Registered Trademark) HTb

A 1916 bp DNA fragment, obtained by digesting with EcoRI and XhoI pGAT1 that had been obtained in Example 2, was isolated and purified, and ligated into the EcoRI/XhoI cloning sites of the gene expression vector pFastBac (Registered Trademark) HTb. Hereinafter, the resulting vector was named pGAT3.

Then, a nucleotide sequence encoding a His-tag was introduced at the 3′-end of the cotton aphid peptidyl-dipeptidase cDNA. That is, 207 bp of a SfuI/XhoI DNA fragment of the clone AC301 was isolated and purified, and ligated into the 6519 bp SfuI/XhoI DNA fragment of the pGAT3. The resulting vector was named pGAT6. The clone AC301 was generated by PCR as follows. First-strand cDNA was synthesized by employing cotton aphid total RNA as a template and using PfuUltra High Fidelity polymerase (manufactured by Stratagene). The PCR was performed according to the manual annexed to the reagent and, as primers, an oligonucleotide comprising the nucleotide sequence of SEQ ID NO: 5 and an oligonucleotide comprising the nucleotide sequence of SEQ ID NO: 6 were used. PCR conditions were 1 cycle of 94° C. for 2 minutes; followed by 35 cycles of PCR, one cycle being 94° C. for 15 seconds, 60° C. for 15 seconds and 72° C. for 25 second; and followed by 1 cycle of 72° C. for 7 minutes. Then, the resulting PCR product was analyzed and purified by agarose gel electrophoresis to obtain the DNA fragment of interest. Further, the obtained DNA fragment was cloned into the pCR-blunt vector (manufactured by Invitrogen), and the nucleotide sequence of the cloned DNA fragment was determined. The obtained plasmid was named AC301.

The N-terminal His-tag of pGAT6 was removed by digestion with RsrI/EcoRI and was replaced with a linker, where the linker was generated by annealing an oligonucleotide comprising the nucleotide sequence of SEQ ID NO: 7 with an oligonucleotide comprising the nucleotide sequence of SEQ ID NO: 8. The obtained vector was a vector in which a nucleotide sequence encoding a full-length cotton aphid peptidyl-dipeptidase A with the C-terminal His-tag is inserted into pFastBac (Registered Trademark) HTb, and was named pGAT14 hereinafter.

### (2) Generation of Recombinant Bacmid DNA

Using the obtained pGAT14, a competent cell of Escherichia coli DH10Bac was transformed. Recombinant bacmid DNA for cotton aphid peptidyl-dipeptidase A was isolated from the transformed Escherichia coli DH10Bac. All procedures were according to the manual annexed to Bac-to-Bac Baculovirus (Trademark) Expression System (manufactured by Invitrogen).

The presence or the absence of the objective gene in the recombinant bacmid was verified by PCR analysis. As the bacmid contains M13 Forward(−40) and M13 Reverse priming sites, the M13Forward(−40) and the M13Reverse primers were used. Also a combination the M13 Forward(−40) or M13 Reverse primers and a primer specific for the insert was also used. Each manipulation was performed according to the manual annexed to Bac-to-Bac Baculovirus (Trademark) Expression System (manufactured by Invitrogen). Each PCR condition was as follows.

(a) In the case of an oligonucleotide comprising the nucleotide sequence of SEQ ID NO: 9 and an oligonucleotide comprising the nucleotide sequence of SEQ ID NO: 10:

(i) 94° C. for 5 minutes; followed by

(ii) 20 cycles of 94° C. for 15 seconds, 60° C. for 15 seconds, and 72° C. for 4 minutes and 30 seconds; followed by

(iii) 25 cycles of 94° C. for 15 seconds, and 63° C. for 15 seconds and 72° C. for 4 minutes and 30 seconds (increase of 5 seconds every +1 cycle); and followed by

(iv) 72° C. for 7 minutes.

(b.) In the case of an oligonucleotide comprising the nucleotide sequence of SEQ ID NO: 10 and an oligonucleotide comprising the nucleotide sequence of SEQ ID NO: 11:

(i) 94° C. for 5 minutes; followed by

(ii) 20 cycles of 94° C. for 15 seconds, 60° C. for 15 seconds, and 72° C. for 4 minutes and 30 seconds; followed by

(iii) 25 cycles of 94° C. for 15 seconds, 63° C. for 15 seconds, and 72° C. for 4 minutes and 30 seconds (increase of 5 seconds every +1 cycle); and followed by

(iv) 72° C., 7 minutes.

Based on the size of the amplified DNA fragments, two kinds of recombinant bacmids of pGAT15 and pGAT16 were selected. DNA fragments amplified from both bacmids had the correct size. Therefore, it was indicated that transposition of the pFastBac (Registered Trademark) expression construct into the bacmid DNA had occurred.

### Example 5

### Preparation of Recombinant Bacmid Stock

### (1) Transfection of Recombinant Bacmid

The recombinant bacmid pGAT15 was transfected into Sf9 cells (ATCC: CRL-1711) in order to generate recombinant baculovirus according to the manual annexed to Bac-to-Bac Baculovirus (Trademark) Expression System (manufactured by Invitrogen). For the transfection, following materials were used: cellfectin (manufactured by Invitrogen), Grace's Insect Cell Culture Medium supplemented with L-amino acids (manufactured by Invitrogen, Gibco), 10% of Foetal Bovine Serum (manufactured by Clontech), and penicillin/streptomycin (manufactured by Life Technologies). 2 μg of bacmid DNA and 7 μl of cellfectin were used. P1 recombinant baculovirus stock was harvested after 8 days according to the manual annexed to Bac-to-Bac Baculovirus (Trademark) Expression System (manufactured by Invitrogen).

### <Large Scale Preparation of Recombinant Baculovirus>

The cotton aphid peptidyl-dipeptidase A baculovirus stock was amplified by inoculating 0.5 ml of P1 virus stock or subsequent generated virus stock on 300 ml of Sf9 insect cell cultures of 1×10⁶ cells/ml. The amplified baculovirus stocks were harvested after 4 days of culture according to the manual annexed to Bac-to-Bac Baculovirus (Trademark) Expression System (manufactured by Invitrogen). The Sf9 cell cultures were grown as suspension in Erlenmeyers (Elscolab) at 135 rpm and at 27° C. The medium components were as follows: Grace's Insect Cell Culture Medium supplemented with L-amino acids (manufactured by Invitrogen, Gibco), 10% Foetal Bovine Serum (manufactured by Clontech), penicillin/streptomycin (manufactured by Life Technologies), and pluronic F-68 solution at final concentration of 0.1% (manufactured by Sigma-aldrich).

The titer of the baculovirus stock was determined by plaque assay according to the manual annexed to Bac-to-Bac Baculovirus (Trademark) Expression System (manufactured by Invitrogen), but for the medium of the plaque assay, 2% agarose was used instead of 4% agarose.

### Example 6

### Expression of Peptidyl-Dipeptidase A in Insect Cell

Sf9 cells of 4×10⁸ were suspended in 30 ml of recombinant baculovirus stock in which the cotton aphid peptidyl-dipeptidase A gene was introduced, and rotation-cultured in a 125 ml Erlenmeyer (manufactured by Elscolab) at 27° C., 135 rpm for 1 hour. Thereafter, the cell suspension was equally distributed over three 250 ml Erlenmeyer (manufactured by Elscolab), and the cell culture medium described in Example 5 was added until a volume of a culturing solution in each Erlenmeyer became 100 ml. The Sf9 cells were rotation-cultured at 135 rpm at 27° C. for 48 hours in the prepared culturing solution. The cultured solution was centrifuged at 1200 rpm to harvest Sf9 cells infected with baculovirus. The harvested Sf9 cells were flash frozen in liquid nitrogen and stored at −80° C. until further use.

### Example 7

### Purification of Peptidyl-Dipeptidase A

### (1) Preparation of Crude Extract

The frozen Sf9 cells infected with baculovirus were re-suspended in 30 ml of buffer A (50 mM Hepes pH 7.5, 0.5 m NaCl, 10 mM imidazole), and subsequently lysed in buffer A by means of a French Press (manufactured by Thermo Spectronic). The pressure was maintained at 1300-1500 psi during the procedure of breaking of the cells. The French pressed solution was centrifuged at 13,000×g at 2° C. for 20 minutes to obtain supernatant. The obtained supernatant was filtered through a 0.45 μm filter and kept on ice.

### (2) Purification

The aphid peptidyl-dipeptidase A was cloned in frame with the 6×His tag in pFastBacHTb. The recombinant protein has been purified using metal affinity chromatography, using either the HiTrap Chelating HP (Amersham biosciences) or HisTrap HP (Amersham biosciences) columns, according to the instructions of the manufacturer (Amersham biosciences). The purification procedure was undertaken on the ÄKTA-FPLC (Amersham biosciences).

The HiTrap/HisTrap affininity columns have been prepared according to the manufacturer's protocol (Amersham biosciences). Buffer A, the binding buffer, was made of 50 mM Hepes pH 7.5, 0.5M NaCl, 10 mM imidazole; and buffer B, the elution buffer, was made of 50 mM Hepes pH 7.5, 0.5M NaCl, 500 mM Imidazole. The purification protocol to purify the aphid peptidyl-dipeptidase A included the following steps:

(i) sample injection,

(ii) washing out unbound protein with 13 column volumes (CV) of buffer A,

(iii) washing for 15 CV with 7% buffer B (35 mM imidazole) for Equilibration of the column,

(iv) elution of purified protein with 6 CV 50% buffer B (250 mM imidazole), and

(v) washing the column with 5 CV 100% buffer B (500 mM imidazole).

The obtained elution fractions were analysed to verify presence of the recombinant cotton aphid peptidy-dipeptidase A by means of standard techniques of SDS-PAGE and western blot. An 8% polyacrylamide gel was used for optimal gel electrophoresis resolution of the peptidyl-dipeptidase A protein, since the molecular weight of the peptidyl-dipeptidase A protein was 75 kDa. For western blot analysis, an anti-His(H15) sc-803 rabbit polyclonal IgG antibody (manufactured by tebubio) was used as primary antibody at a 1:500 dilution. The secondary antibody was a goat anti-rabbit-HRP (manufactured by Pierce) at a dilution of 1:10000.

After analysis of the gels by SDS-PAGE and Western blotting, the fractions of interest were pooled and glycerol was added to a final concentration of 10%. The protein concentration was determined with the Bradford spectro-photometric protocol using Pre-diluted Protein Assay Standards: Bovine Serum Albumin Fraction V Set according to the manufacturer's protocol (Bio-Rad). The pooled fractions were then distributed into several aliquots and immediately flash-frozen in liquid nitrogen and stored at −80° C.

### (3) Desalting

The imidazole in the purified cotton aphid peptidyl-dipeptidase A fraction was removed by buffer exchange using 5 ml HiTrap desalting columns (manufactured by Amersham biosciences). The buffer exchange procedure was undertaken on the ÄKTA-FPLC (Amersham biosciences) as follows. Buffer C, the equilibration and elution buffer, was made of 50 mM Hepes pH 7.5 and 0.5M NaCl. The protocol to remove imidazole from the cotton aphid peptidyl-dipeptidase A fraction comprised the following steps:

(i) injection of 1.5 ml sample

(ii) equilibration of column with 4.5 ml of buffer C,

(iii) elution of purified protein with 9 ml buffer C, and

(iv) washing the column with 20 ml buffer C.

The obtained elution fraction was analyzed to verify the presence of the recombinant cotton aphid peptidyl-dipeptidase A by means of standard techniques of SDS-PAGE and western blot as described above. The protein concentration was determined with the Bradford spectro-photometric protocol using Pre-diluted Protein Assay Standards: Bovine Serum Albumin Fraction V Set according to the manufacturer's protocol (Bio-Rad). The elution fraction was supplemented with glycerol (10% final concentration), flash-frozen in liquid nitrogen and stored at −80° C.

### Example 8

### Selection of Compound which Inhibits Peptidyl-Dipeptidase A Activity

Selection of a compound which modulates the activity of a peptidyl-dipeptidase A was performed in a system where the activity of a peptidyl-dipeptidase A which is modulated by adding a test compound to an in vitro reaction system using the aphid peptidyl-dipeptidase A prepared in Example 7 is measured and evaluated.

Measurement of the activity of the aphid peptidyl-dipeptidase A was performed according to a method using Abz-Gly-p-nitro-Phe-Pro-OH (manufactured by Bachem, M-1100) as a substrate, as described in the previously reported article (Carmel et al. (1979) Clinica Chimica Acta, 93, 215-220).

For measuring the activity, the activity of the aphid peptidyl-dipeptidase A was measured when a test compound dissolved in DMSO was contained to a final concentration of 10 μM. In addition, the activity of the aphid peptidyl-dipeptidase A was measured when DMSO was contained in place of a test compound. Then, a ratio (%) of a measured value of the activity of the aphid peptidyl-dipeptidase A when a test compound dissolved in DMSO was contained, relative to a measured value of the activity of aphid peptidyl-dipeptidase A when DMSO was contained in place of the test compound was calculated, and a value obtained by subtracting the calculated value from 100% was adopted as an inhibition degree (%). Results in each test compound are shown in Table 4 in Example 9 together with results of Example 9.

The activity of aphid peptidyl-dipeptidase A was measured when a test compound dissolved in DMSO was contained to a final concentration of each concentration of 100 μM, 30 μM, 10 μM, 3 μM, 1 μM, 0.3 μM, 0.1 μM or 0.03 μM. IC₅₀ (μM) was calculated from the result of each concentration at each test compound using a concentration-dependent test analyzing software XL fit (manufactured by idbs). Results are shown in Table 5 in Example 10 together with results of Example 9.

### Example 9

### Pesticidal Activity Test

A sterilized artificial feed having the following composition (Table 3) was prepared. Then, according to the same manner as that of the method described in Handbook of Insect Rearing Vol. 1 (Elsevier Science Publisers 1985) pp. 35 to pp. 36 except that a test compound dissolved in DMSO to a final concentration of 640 μM was added at 0.5% volume of the artificial feed, and components were mixed, Aphis gossypil was reared. Six days after rearing, the number of surviving Aphis gossypil was investigated, and an entity exhibiting a significant controlling value (e.g. controlling value of 30% or more) was determined to have pesticidal activity by obtaining a controlling value by the following equation.

Controlling value (%)={1−(Cb×Tai)/(Cai×Tb)}×100

Letters in the equation represent the following meanings.

Cb: Number of surviving worms before treatment in non-treated section'
Cai: Number of surviving worms at observation in non-treated section
Tb: Number of surviving worms before treatment in treated section
Tai: Number of surviving worms at observation in treated section

Results are shown in Table 4 in Example 9 together with results of Example 8.

TABLE 3

| | (mg/100 ml) |
|---|---|
| Amino acid | |
| L-alanine | 100.0 |
| L-arginine | 275.0 |
| L-asparagine | 550.0 |
| L-aspartic acid | 140.0 |
| L-cysteine | 40.0 |
| (hydrochloride) | |
| L-glutamic acid | 140.0 |
| L-glutamine | 150.0 |
| L-glycine | 80.0 |
| L-histidine | 80.0 |
| L-isoleucine | 80.0 |
| L-leucine | 80.0 |
| L-lysine | 120.0 |
| (hydrochloride) | |
| L-methionine | 80.0 |
| L-phenylalanine | 40.0 |
| L-proline | 80.0 |
| L-serine | 80.0 |
| L-threonine | 140.0 |
| L-tryptophan | 80.0 |
| L-tyrosine | 40.0 |
| L-valine | 80.0 |
| Vitamins | |
| Ascorbic acid | 100.0 |
| Biotin | 0.1 |
| Calcium | 5.0 |
| pantothenate | |
| Choline chloride | 50.0 |
| Inositol | 50.0 |
| Nicotinic acid | 10.0 |
| Thiamine | 2.5 |
| Others | |
| Sucrose | 12500.0 |
| Dipotassium | 1500.0 |
| hydrogen phosphate | |
| Magnesium sulfate | 123.0 |
| Cupric chloride | 0.2 |
| Ferric chloride | 11.0 |
| Manganese chloride | 0.4 |
| Zinc sulfate | 0.8 |
| (anhydrous) | |

Adjusted to pH 6.8

TABLE 4

| | | Activity of | |
|---|---|---|---|
| | | inhibiting | |
| | | peptidyl- | |
| | | dipeptidase A | Determination |
| | | activity (inhibition | result of |
| Compound | | degree (%) at 10 | pesticidal |
| name | Structure | μM addition) | activity |
| captopril | 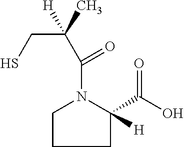 | 99 | Presence of pesticidal activity |
| MMP-2/MMP-3 inhibitor I | 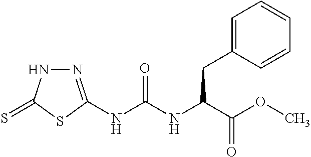 | 79 | Presence of pesticidal activity |
| benazepril | 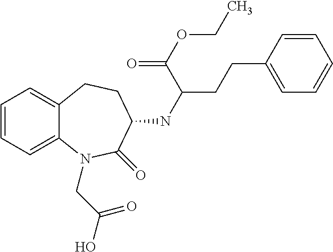 | 41 | Presence of pesticidal activity |
| 3-beta-[2- (diethylamino) ethoxy]androst- 5-en-17-one | 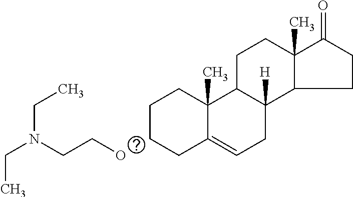 | 17 | Presence of pesticidal activity |
| enalapril | 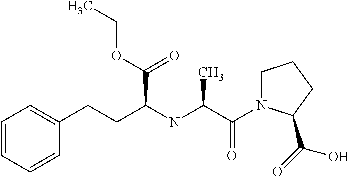 | 98 | Presence of pesticidal activity |
| ramipril | 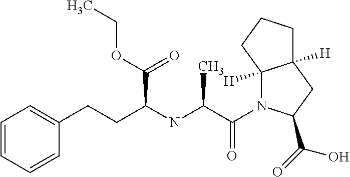 | 16 | Presence of pesticidal activity |
| quinapril | 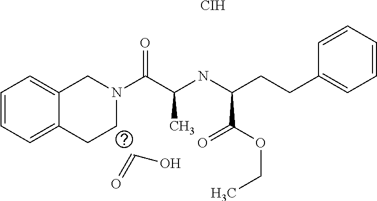 | 15 | Presence of pesticidal activity |

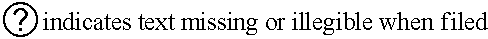

### Example 10

### Pesticidal Activity Test

According to the same manner as that of Example 9 except that a test compound dissolved in DMSO to a final concentration of 50 ppm was added, pesticidal activity test was performed, and results are shown in Table 5 in Example 10 together with results of Example 8.

TABLE 5

| | | Activity of | |
|---|---|---|---|
| | | inhibiting | |
| | | peptidyl- | Determination |
| | | dipeptidase | result of |
| Compound | | A activity | pesticidal |
| name | Structure | IC50, (μM)) | activity |
| benazepril | 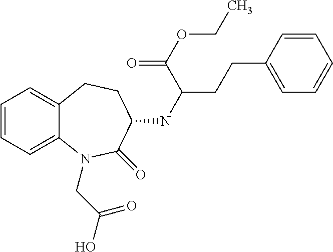 | 15.9 | Presence of pesticidal activity |
| quinapril | 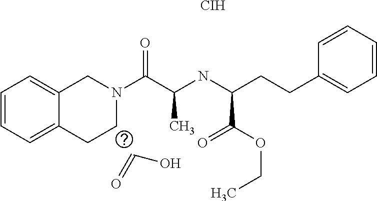 | 35.8 | Presence of pesticidal activity |
| DE00132 | 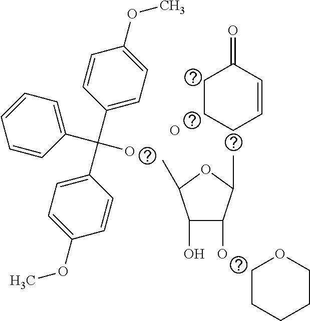 | >100 | Absence of pesticidal activity |
| DE05553 | 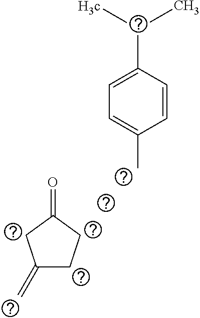 | >100 | Absence of pesticidal activity |
| DF15164 | 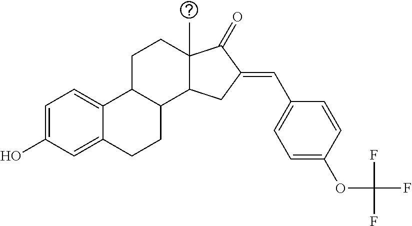 | >100 | Absence of pesticidal activity |

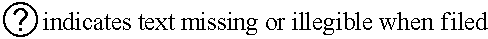

### INDUSTRIAL APPLICABILITY

According to the present invention, it becomes possible to provide a more target-based approach of screening agricultural chemicals, whereby compounds are screened against a specific target that has been identified as biologically and/or physiologically relevant with intent of chemically interfering with the target site to control insects or other pest organisms.

### Free Text in Sequence Listing

### SEQ ID NO: 3

Designed oligonucleotide primer for PCR

### SEQ ID NO: 4

Designed oligonucleotide primer for PCR

### SEQ ID NO: 5

Designed oligonucleotide primer for PCR

### SEQ ID NO: 6

Designed oligonucleotide primer for PCR

### SEQ ID NO: 7

Designed oligonucleotide linker for ligation

### SEQ ID NO: 8

Designed oligonucleotide linker for ligation

### SEQ ID NO: 9

Designed oligonucleotide primer for PCR

### SEQ ID NO: 10

Designed oligonucleotide primer for PCR

### SEQ ID NO: 11

Designed oligonucleotide primer for PCR

1. An agent that modulates physiological condition of pests, wherein the agent has an ability to modulate the activity of an insect peptidyl-dipeptidase A.

2. An agent according to claim 1, wherein the peptidyl-dipeptidase A is a cotton aphid peptidyl-dipeptidase A.

3. An agent according to claim 1, wherein the agent is a pesticidal agent.

4. An agent according to claim 1, wherein the ability to modulate the activity of an insect peptidyl-dipeptidase A is an ability to inhibit a reaction of the insect peptidyl-dipeptidase A with o-aminobenzoylglycyl-p-nitro-L-phenylalanyl-L-proline.

5. A pesticidal agent which comprises a substance that has an ability to modulate the activity of an insect peptidyl-dipeptidase A or an agriculturally acceptable salt of the substance as an active ingredient.

6. A pesticidal agent according to claim 5, wherein the substance has an ability to inhibit a reaction of the insect peptidyl-dipeptidase A with o-aminobenzoylglycyl-p-nitro-L-phenylalanyl-L-proline.

7. A pesticidal agent according to claim 6, wherein the substance has an ability to inhibit the reaction of the insect peptidyl-dipeptidase A with o-aminobenzoylglycyl-p-nitro-L-phenylalanyl-L-proline in a cell-free system, wherein in the presence of the substance of 10 μM or more the activity of the peptidyl-dipeptidase A is lower than that in the absence of the substance.

8. A pesticidal agent according to claim 6, wherein the substance has an ability to inhibit a reaction of the insect peptidyl-dipeptidase A with o-aminobenzoylglycyl-p-nitro-L-phenylalanyl-L-proline in a cell-free system with an IC50 of 100 μM or less.

9. A method for assaying pesticidal activity of a test substance, which comprises:
(1) a first step of measuring the activity of a peptidyl-dipeptidase A selected from the following group A in a reaction system in which the peptidyl-dipeptidase A contacts with a test substance; and
(2) a second step of evaluating the pesticidal activity of the test substance based on the difference obtained by comparing the activity measured in the first step with the activity of a control:
<Group A>
(a) a protein comprising the amino acid sequence of SEQ ID NO: 1;
(b) a protein comprising an amino acid sequence with deletion, addition or substitution of one or more amino acids in the amino acid sequence of SEQ ID NO: 1, wherein the protein has peptidyl-dipeptidase A activity;
(c) a protein comprising an amino acid sequence that has sequence identity of 65% or more to the amino acid sequence of SEQ ID NO. 1, wherein the protein has peptidyl-dipeptidase A activity;
(d) a protein comprising the amino acid sequence encoded by the nucleotide sequence of SEQ ID NO: 2;
(e) a protein comprising an amino acid sequence encoded by a nucleotide sequence that has sequence identity of 65% or more to the nucleotide sequence of SEQ ID NO: 2, wherein the protein has peptidyl-dipeptidase A activity;
(f) a protein comprising an amino acid sequence encoded by a polynucleotide, wherein the polynucleotide hybridizes under a stringent condition to a polynucleotide comprising a nucleotide sequence complementary to the nucleotide sequence of SEQ ID NO: 2, and wherein the protein has peptidyl-dipeptidase A activity;
(g) a protein comprising an amino acid sequence of an insect peptidyl-dipeptidase A; and
(h) a protein comprising an amino acid sequence of a cotton aphid peptidyl-dipeptidase A.

10. A method for screening a pesticidal substance, which comprises selecting a substance having the pesticidal activity that is evaluated by the method according to claim 9.

11. A pesticidal agent which comprises a substance selected by the method according to claim 10 or agriculturally acceptable salts thereof as an active ingredient.

12. A method for controlling pests which comprises applying an effective amount of the pesticidal agent according to claim 5, 6, 7, 8 or 11 to the pest, habitat of the pest or plant to be protected from the pest.

13. A method for controlling pests which comprises:
identifying a substance having the pesticidal activity that is evaluated by the method according to claim 9, and
contacting the pest with the identified pesticidal substance.

14. An insect peptidyl-dipeptidase A comprising an amino acid sequence selected from the following group B:
<Group B>
(a) the amino acid sequence of SEQ ID NO; 1;
(b) an amino acid sequence with deletion, addition or substitution of one or more amino acids in the amino acid sequence of SEQ ID NO: 1, wherein the amino acid sequence has peptidyl-dipeptidase A activity;
(c) an amino acid sequence that has sequence identity of 65% or more to the amino acid sequence of SEQ ID NO: 1, wherein the amino acid sequence has peptidyl-dipeptidase A activity;
(d) the amino acid sequence encoded by the nucleotide sequence of SEQ ID NO: 2;
(e) an amino acid sequence encoded by a nucleotide sequence that has sequence identity of 65% or more to the nucleotide sequence of SEQ ID NO: 2, wherein the amino acid sequence has peptidyl-dipeptidase A activity;
(f) an amino acid sequence encoded by a polynucleotide, wherein the polynucleotide hybridizes under a stringent condition to a polynucleotide comprising a nucleotide sequence complementary to the nucleotide sequence of SEQ ID NO: 2, wherein the amino acid sequence has peptidyl-dipeptidase A activity; and
(g) an amino acid sequence of a cotton aphid peptidyl-dipeptidase A.

15. Use of an insect peptidyl-dipeptidase A as a reagent that provides an indicator to evaluate pesticidal activity.

16. Use of an Insect peptidyl-dipeptidase A according to claim 14 as a reagent that provides an indicator to evaluate pesticidal activity.

17. A polynucleotide which comprises a nucleotide sequence encoding an amino acid sequence of a peptidyl-dipeptidase A according to claim 14.

18. A polynucleotide according to claim 17, which comprises the nucleotide sequence of SEQ ID NO: 2.

19. A polynucleotide which comprises a nucleotide sequence 25 complementary to a nucleotide sequence of a polynucleotide according to claim 17 or 18.

20. A polynucleotide which comprises:
a partial nucleotide sequence of a polynucleotide according to claim 17 or 18; or
a nucleotide sequence complementary to the partial nucleotide sequence.

21. A polynucleotide according to claim 20, which comprises a nucleotide sequence of SEQ ID NO: 3 or 4.

22. A method for obtaining a polynucleotide comprising a nucleotide sequence encoding an amino acid sequence of a peptidyl-dipeptidase A, which comprises:
a step of amplifying a desired polynucleotide by polymerase chain reaction using as a primer a polynucleotide according to claim 20;
a step of identifying the desired polynucleotide amplified; and
a step of recovering the identified polynucleotide.

23. A method for obtaining a polynucleotide comprising a nucleotide sequence encoding an amino acid sequence of a peptidyl-dipeptidase A, which comprises:
a step of detecting a desired polynucleotide by hybridization using as a probe a polynucleotide according to claim 19:
a step of identifying the desired polynucleotide detected; and
a step of recovering the identified polynucleotide.

24. A circular polynucleotide comprising a nucleotide sequence of a polynucleotide according to claim 17 or 18, wherein the nucleotide sequence is operably linked to a baculovirus promoter.

25. A circular polynucleotide according to claim 24, wherein the promoter is a polyhedrin gene promoter.

26. A circular polynucleotide according to claim 24, wherein the polynucleotide comprises a replication origin for autonomous replication in a host cell.

27. A circular polynucleotide according to claim 24, wherein the polynucleotide comprises a nucleotide sequence of a baculovirus shuttle vector and is capable of propagating as a virus in an insect cell.

28. A method for producing a circular polynucleotide, which comprises ligating a polynucleotide according to claim 17 or 18 into a vector.

29. A transformant in which a polynucleotide according to claim 17 or 18 is introduced.

30. A transformant according to claim 29, wherein the transformant is a transformed insect cell.

31. A method for producing a transformant, which comprises introducing a polynucleotide according to claim 17 or 18 into a host cell.

32. A recombinant baculovirus comprising within its genome a polynucleotide according to claim 17 or 18.

33. A method for producing a peptidyl-dipeptidase A, which comprises a step of culturing the transformant according to claim 29 and recovering a produced peptidyl-dipeptidase A.

34. Use of a peptidyl-dipeptidase A comprising an amino acid sequence selected from the following group B:
<Group B>
(a) the amino acid sequence of SEQ ID NO; 1;
(b) an amino acid sequence with deletion, addition or substitution of one or more amino acids in the amino acid sequence of SEQ ID NO: 1, wherein the amino acid sequence has peptidyl-dipeptidase A activity;
(c) an amino acid sequence that has sequence identity of 65% or more to the amino acid sequence of SEQ ID NO: 1, wherein the amino acid sequence has peptidyl-dipeptidase A activity;
(d) the amino acid sequence encoded by the nucleotide sequence of SEQ ID NO: 2;
(e) an amino acid sequence encoded by a nucleotide sequence that has sequence identity of 65% or more to the nucleotide sequence of SEQ ID NO: 2, wherein the amino acid sequence has peptidyl-dipeptidase A activity;
(f) an amino acid sequence encoded by a polynucleotide, wherein the polynucleotide hybridizes under a stringent condition to a polynucleotide comprising a nucleotide sequence complementary to the nucleotide sequence of SEQ ID NO: 2, wherein the amino acid sequence has peptidyl-dipeptidase A activity; and
(g) an amino acid sequence of a cotton aphid peptidyl-dipeptidase A or a polynucleotide according to claim 17 as a research tool.

35. Use according to claim 34, wherein the research tool is an experimental tool for screening a pesticidal substance.

36. A system which comprises:
a means to input, store and manage a data information of an ability of test substances, wherein the ability is an ability to modulate the activity of an insect peptidyl-dipeptidase A;
a means to query and retrieve the data information based on a desired criterion; and
a means to display and output the result which is queried and retrieved.
